(19) United States
(12) Patent Application Publication
(10) Pub. No.: US 2025/0206779 A1
(43) Pub. Date: Jun. 26, 2025

(54) ANGIOTENSIN-(1-9) ANALOGUE BASED ON D AMINO ACIDS, PHARMACEUTICAL COMPOSITIONS AND USES THEREOF

(71) Applicants: UNIVERSIDAD DE CHILE, Santiago (CL); PONTIFICIA UNIVERSIDAD CATÓLICA DE CHILE, Santiago (CL)

(72) Inventors: María Paz OCARANZA GERALDINO, Santiago (CL); Marcelo Javier KOGAN, Santiago (CL); Sergio Alejandro LAVANDERO GONZÁLEZ, Santiago (CL); Mario MartÍn CHIONG LAY, Santiago (CL)

(21) Appl. No.: 18/560,544

(22) PCT Filed: May 13, 2021
(86) PCT No.: PCT/IB2021/054074

§ 371 Date: Nov. 13, 2023

(51) Int. Cl. C07K 7/14; A61K 38/08; A61K 45/06; A61P 9/00; A61P 9/12

(52) U.S. Cl. CPC C07K 7/14; A61K 38/085; A61K 45/06; A61P 9/00; A61P 9/12

(57) ABSTRACT

The present invention provides an angiotensin-(1-9) analog, particularly an angiotensin-(1-9) peptide synthesized from D-amino acids. This invention further involves the inversion of the original sequence of angiotensin-(1-9). This analog possesses an increased stability and maintains the biological activity of angiotensin-(1-9). Another embodiment of this invention comprises pharmaceutical compositions containing said analog and their use in the treatment of cardiovascular diseases, tissue remodeling in kidney, brain, and also the induction of cardioprotection.

### FIELD OF INVENTION

The present invention is focused on the field of the renin-angiotensin system, in particular, the angiotensin-(1-9) peptide. In one particular embodiment, the present invention provides angiotensin-(1-9) analogs. The angiotensin-(1-9) analogs of this invention are related to a peptide formed by D-amino acids of the same angiotensin-(1-9) sequence, but in an inverted order. The carboxyl terminal of said analog may be free or amidated. Another embodiment provides pharmaceutical compositions containing such analogs and methods to use the same for the treatment of cardiovascular, renal, and cerebral, diseases, and to provide cardioprotective and anti-remodeling effects in patients and animals.

### BACKGROUND OF THE INVENTION

Angiotensins are peptides derived from angiotensinogen. These peptides are:

Angiotensinogen*:
(SEQ ID NO: 1)
Asp-Arg-Val-Tyr-Ile-His-Pro-Phe-His-Leu-
Leu-Val-Tyr-Ser
Angiotensin I:
(SEQ ID NO: 2)
Asp-Arg-Val-Tyr-Ile-His-Pro-Phe-His-Leu
Angiotensin II:
(SEQ ID NO: 3)
Asp-Arg-Val-Tyr-Ile-His-Pro-Phe
Angiotensin III:
(SEQ ID NO: 4)
Arg-Val-Tyr-Ile-His-Pro-Phe
Angiotensin-IV:
(SEQ ID NO: 5)
Val-Tyr-Ile-His-Pro-Phe
Angiotensin-(1-9):
(SEQ ID NO: 6)
Asp-Arg-Val-Tyr-Ile-His-Pro-Phe-His
Angiotensin-(1-7):
(SEQ ID NO: 7)
Asp-Arg-Val-Tyr-Ile-His-Pro
* The first amino acid of the sequences
corresponds to the amino terminal end.

Several physiological and biological functions have been described for the various angiotensins.

Des-aspartate-angiotensin I has been described for use in the treatment and/or prevention of cardiac hypertrophy (U.S. Pat. No. 5,773,415) and formation of neointima or restenosis (U.S. Pat. No. 6,100,237).

Angiotensin II (SEQ ID NO: 3) is involved in cardiac hypertrophy and neointima formation. Exogenous administration of angiotensin II enhances cardiac hypertrophy (Dostal & Baker, Am. J Hypertens. 5: 276-80, 1991) and neointima formation (Osterrieder et al., Hypertension 18: 1160-4, 1991; Daemen et al., Circ Res. 68: 450-6, 1991).

Angiotensin Ill (SEQ ID NO: 4) induces natriuresis in an AT2 receptor dependent mechanism. Moreover, this peptide also induces vasoconstriction and aldosterone release (Fyhrquist & Saijonmaa, J Intern Med. 264: 224-36, 2008).

Angiotensin IV (SEQ ID NO: 5), a secondary metabolite of angiotensin 1l, has antihypertrophic actions and also inhibits the neointima formation (EP1846017).

Angiotensin-(1-7) (SEQ ID NO: 7) has opposite actions to angiotensin 1l. This peptide induces vasodilation and has antihypertensive and antifibrotic actions (Katovich et al., Curr Hypertens Rep. 10: 227-32, 2008).

Angiotensin-(1-9) (SEQ ID NO: 6) is synthesized by hydrolysis of the terminal amino acid leucine of angiotensin I by the analogous angiotensin-converting enzyme (ACE2) (Donoghue et al., J Mol Cell Cardiol. 35: 1043-53, 2003). Subsequently, angiotensin-(1-9) is degraded by the angiotensin converting enzyme (ACE1) to angiotensin-(1-7). Like angiotensin-(1-7), angiotensin-(1-9) has opposite actions to that of angiotensin II (Ocaranza et al, J Hypertens. 28: 1054-64, 2010).

Angiotensin-(1-9) decreases blood pressure and reduces cardiovascular damage in three experimental hypertensive models: angiotensin II infusion model with minipumps, Goldblatt 2K-1C model, and DOCA salt model. Chronic administration of angiotensin-(1-9) to hypertensive rats reduced systolic blood pressure, improved cardiac and endothelial function as well as cardiovascular remodeling and oxidative stress (Ocaranza et al., J Hypertens. 32: 771-83, 2014). Angiotensin-(1-9) also attenuates fibrosis in spontaneously hypertensive rats (Flores-Munoz et al., J Physiol. 589: 939-51, 2011). The administration of angiotensin-(1-9) by using mini osmotic pumps to infarcted rats by ligation of the left coronary artery, prevented cardiac hypertrophy, evaluated by the decrease in the following markers: ANF (atrial natriuretic factor) mRNA levels, β-MHC (beta myosin heavy chain) protein levels and cardiomyocyte size (area and perimeter) (Ocaranza et al., J Hypertens. 28: 1054-64, 2010). Other studies indicate that angiotensin-(1-9) favors bradykinin binding to its B2 receptor probably due to conformational changes in the ACE-B2 receptor complex (Erdos et al., J Mol Cell Cardiol. 34: 1569-76, 2002).

We have previously filed two patent applications: CL2008003736 and CL2010000950. The patent application CL2008003736 corresponds to a pharmaceutical composition comprising an effective amount of angiotensin-(1-9) and at least one pharmaceutically acceptable carrier, excipient, stabilizer, diluent and/or adjuvant. Furthermore, said invention describes the use of angiotensin-(1-9) pharmaceutical compositions useful for preventing, reversing, inhibiting and/or reducing cardiovascular, pulmonary, cerebral, or renal remodeling. In addition, the application CL2008003736 also comprises a method to prevent, reverse, inhibit and/or decrease cardiovascular, pulmonary, cerebral, or renal remodeling that consists in the elevation of angiotensin-(1-9) concentration in the blood and/or tissues by means of a pharmaceutical composition containing a vector that expresses ACE2, enzyme responsible for the endogenous production of angiotensin-(1-9). These vectors correspond to adenoviruses, retroviruses, lentiviruses, or adeno-associated viruses that contain the ACE2 gene. The application CL2008003736 discloses the administration of angiotensin-(1-9) by oral, injectable, and continuous infusion using pumps. This patent application further provides a method to increase angiotensin-(1-9) levels in the body by treatment of patients with angiotensin-I converting enzyme inhibitors, with angiotensin II receptor antagonists (ARA II), with Rho kinase inhibitors, with L calcium channel blockers and/or with diuretics. Patent application CL2010000950 describes the use of angiotensin-(1-9) to control blood pressure and/or vasculature dilation, and further discloses medical use of such peptide comprising by the administration of angiotensin-(1-9) for the treatment of hypertension, and as an agent to induce vasodilation.

Although the peptides present a high bioactivity with a high specificity with few or no side effects, as is the case of angiotensin-(1-9), most peptides are short-lived molecules, and are easily degraded by enzymes, with little or no therapeutic use (Liu et al., Chem Rec. 16: 1772-86, 2016). The high number of peptidases present in the blood is responsible for their very short half-life in the plasma, normally less than one minute (Segura-Campos et al., Rev Chil Nutr. 37: 386-91, 2010). The strategy for converting these molecules into useful drugs often involves transforming them into peptidomimetics. One strategy for designing protease stable peptides is the synthesis of retro-inverse analogues. This strategy consists of using the non-natural D amino acids and reversing the order of the amino acid sequence with respect to the original peptide. In theory, this strategy originates a peptide that contains side chain orientations very similar to that of the original structure (Van Regenmortel & Muller, Curr Opin Biotechnol. 9: 377-82, 2005). The non-natural D-amino acids represent conformational reflex images of natural L-amino acids that are found in all proteins present in biological systems. If properly designed, the retro-inverse peptides can have similar receptor binding characteristics as compared to the natural L-amino acid-based peptides. Depending on the nature of the amino acids, structural stability, spatial orientation, topology of the side chain could be maintained and, consequently, the bioactivity of the peptide. Moreover, the use of non-natural amino acids further provides to the retro-inverse peptides the ability to resist enzymatic degradation (Chorev & Goodman, Trends Biotechnol, 13: 438-445, 1995). Therefore, peptides containing D-amino acids are less susceptible to proteolytic degradation and have a longer half live when used as pharmaceuticals (EP0127234, EP0127235, GB2166139).

Because the complex nature of the spatial topology of the amino acids side chains, not all the retro-inverse peptides maintain the biological activity of the original peptide. The retro-inverse peptide, depending on its side chain topology, can present inherent differences at secondary and tertiary structure level (Li et al., J Biol Chem. 285: 19572-81, 2010). This strategy can also generate inverted peptide bonds with respect to the native peptide, which could affect the binding of this peptide with its receptor, therefore affecting its bioactivity (Fischer, Curr Protein Pept Sci. 4: 339-56, 2003). Another limitation that could affect its bioactivity is the presence of proline in the amino acid chain of angiotensin-(1-9). Proline is an amino acid considered incompatible with this retro-inverse methodology, because its side chain is linked to the central chain, so that by transforming this amino acid to a retro-inverse, the side chain does not remain exactly in the same disposition, as happens with the other side chains. This alteration affects or even nullifies the bioactivity (Fischer, Curr Protein Pept Sci. 4: 339-56, 2003). Example of retro-inverse peptides that did not maintain their original activity correspond to end-capped p53 (15-29) (SQETFSDLWKLLPEN) and RI-p53 (15-29) (^{D}N^{D}E^{D}P^{D}L^{D}L^{D}K^{D}W^{D}L^{D}D^{D}S^{D}F^{D}T^{D}E^{D}Q^{D}S), PMI (TSFAEYWNLLSP) and RI-PMI (^{D}P^{D}S^{D}L^{D}L^{D}N^{D}W^{D}Y^{D}E^{D}A^{D}D^{D}S^{D}T), CAI (ITFEDLLDYYGP) and RI-CAI (^{D}P^{D}G^{D}Y^{D}Y^{D}D^{D}L^{D}L^{D}D^{D}E^{D}F^{D}T^{D}I) and Y4WP40 (APTWSPPPPP) and RI-Y4W-P40 (^{D}P^{D}P^{D}P^{D}P^{D}P^{D}S^{D}W^{D}T^{D}P^{D}A) (Li et al., J Biol Chem. 285: 19572-81, 2010). Using surface plasmon resonance and isothermal titration calorimetry to evaluate the binding of the peptides to their target proteins, RI-p53 (15-29) has between 280- and 306-fold reduction as compared to p53 (15-29). Similar results were obtained for PMI and RI-PMI, CAI and RICAI, and Y4W-P40 and RI-Y4W-P40. All these retro-inverse peptides show a significantly lower binding than the original peptides to the target proteins (Li et al., Bioorg Med Chem. 21: 4045-50, 2013). In addition, retro-inverse peptides of the beta-amyloid protein do not induce anti-beta-amyloid cross-immune response, suggesting that the three-dimensional structure of retro-inverse beta-amyloid peptides are different from the original L-peptides (Bianchi et al., Adv Exp Med Biol. 611: 363-4, 2009).

There are two retro-inverse peptides related to the renin-angiotensin system. The retro-inverse bradykinin showed a Kd to the bradykinin B2 receptor 40 times lower than the native peptide (Xie et al., Cancer Lett 2015, 369: 144-51, 2015). The retro-inverse of the receptor binding epitope of the AT1R activating autoantibodies is capable to block the vasoconstriction of arterioles induced by AT1R activating antibodies (Li et al., Hypertension 65: 793-9, 2015).

Finally, a pseudo retro-inverse peptide from an analogous Angiotensin-(1-7) was described (U.S. Pat. No. 9,670,251). In this patent, Angiotensin-(1-7) sequence (NH₂-DRVYIHP—COOH) was modified by replacing the first amino acid to alanine and the seven amino acid to serine. In the patent, they showed that the peptide NH₂-DRVYIHS—COOH has biological activity comparable to Angiotensin-(1-7). Moreover, they disclosed derivative Angiotensin-(1-7) retro-inverse peptides NH₂—S^{D}H^{D}I^{D}Y^{D}V^{D}R^{D}D^{D}-COOH and NH₂—S^{D}H^{D}I^{D}Y^{D}V^{D}R^{D}V^{D}A^{D}-COOH. However, no examples showing the biological activity of these peptides are presented. On the other hand, these pseudo retro-inverse peptides do not contain proline in the sequence. As explained above, state of the art in retro-inverse peptides predicts that the presence of proline will disrupt biological activity. Hence, in the claimed pseudo retro-inverse peptides, the proline was replaced by serine. In the retro-inverse peptide of our invention, the proline in the sequence is maintained. Surprisingly, the Ang-(1-9) retro-inverse peptide of this invention retains biological activity.

### SUMMARY OF THE INVENTION

The present invention provides angiotensin-(1-9) analogs whereas such analogs are retro-inverse peptides. In one embodiment, the peptide comprises or consists of the amino acid sequence HFPHIYVRD (SEQ ID NO: 9), wherein the peptide comprises one or more D-amino acid residues. In another embodiment, this invention also provides pharmaceutical compositions containing such retro-inverse peptides. This invention further discloses a method to induce cardioprotective and anti-remodeling effects and a method for treating cardiovascular, renal, and cerebral diseases using said retro-inverse peptides.

### BRIEF DESCRIPTION OF THE DRAWINGS

FIG. 1. Analysis of purity and identity of chemically synthesized retro-inverse angiotensin-(1-9). FIG. 1A: Chromatogram of retro-inverse angiotensin-(1-9) obtained by HPLC using photodiode array detector (PDA) showing >98% purity. FIG. 1B: Mass spectrometry analysis of retro-inverse angiotensin-(1-9) confirming identity of the peptide.

FIG. 2. Stability of retro-inverse angiotensin-(1-9) in human serum determined by HPLC analysis. FIG. 2A: Angiotensin-(1-9) before (continuous line) and after 3 h incubation (slashed line) with human serum. FIG. 2B: Retro-inverse angiotensin-(1-9) before (continuous line) and after 48 h incubation (slashed line) with human serum. FIG. 2C: Quantitation of the remaining retro-inverse angiotensin-(1-9) during the incubation with human serum. FIG. 2D Quantitation of the remaining retro-inverse angiotensin-(1-9) amide during the incubation with human serum. *p<0.05 vs TO.

FIG. 3. Effect of retro-inverse angiotensin-(1-9) on cardiomyocyte hypertrophy. Cardiomyocytes were treated with norepinephrine (NE, hypertrophy inductor) in the presence or absence of retro-inverse angiotensin-(1-9) (1, 10 and 100 μM) FIG. 3A: Representative western blot showing the hypertrophic markers beta-myosin heavy chain (β-MHC) and atrial natriuretic peptide (ANP). β-tubulin was loading control. FIG. 3B: Quantitation of ANP protein levels. FIG. 3C: Quantitation of β-MHC protein levels. FIG. 3D: The same experiment as above but using retro-inverse angiotensin-(1-9) amide. Only ANP results is shown. *p<0.05 vs control; #p<0.05 and ##p<0.01 vs respective control with retro-inverse angiotensin-(1-9) (RI-(1-9)).

FIG. 4. Effect of retro-inverse angiotensin-(1-9) on cardiomyocyte hypertrophy. Cardiomyocytes were treated with norepinephrine (NE, hypertrophy inductor) in the presence or absence of retro-inverse angiotensin-(1-9) 100 μM. Cells were stained using phalloidin (actin, red) and Hoechst (nuclei, blue). FIG. 4A: Representative confocal images. FIG. 4B: Quantitation of cell areas. FIG. 4C Quantitation of cell perimeters. ***p<0.001 vs control; #p<0.05 and ##p<0.001 vs NE.

FIG. 5. Effect of retro-inverse angiotensin-(1-9) on fibroblast proliferation. Fibroblast were cultivated in the presence or absence of retro-inverse angiotensin-(1-9). After 48 h of culture, cells were detached by trypsin-EDTA treatment and counted in a Neubauer chamber.

FIG. 6. Effect of retro-inverse angiotensin-(1-9) on cardiomyocyte death. Cardiomyocytes were treated with simulated ischemia/reperfusion (I/R) in the presence or absence of retro-inverse angiotensin-(1-9) 100 μM. FIG. 6A: Lactic dehydrogenase (LDH) release to the culture medium was used as a marker of necrosis. FIG. 6B: Caspase 3 cleavage was used as a marker of apoptosis. ***p<0.001 vs control without I/R treatment; ##p<0.01 and ##p<0.001 vs I/R without retro-inverse angiotensin-(1-9) treatment.

FIG. 7. Effect of retro-inverse angiotensin-(1-9) on myocardial infarct. Isolated hearts in a Langendorff system were submitted to total ischemia for 30 min followed by 120 min reperfusion in the presence or absence of retro-inverse angiotensin-(1-9). Infarcted regions were identified by incubation of sliced heart to 2,3,5-triphenyltetrazolium chloride.

FIG. 8. Effect of retro-inverse angiotensin-(1-9) on blood pressure in spontaneously hypertensive rats (SHR). At week 2, SHR were treated with vehicle (-▪-) [n=12], angiotensin-(1-9) 300 ng/kg/min (-▴-) [n=12], angiotensin-(1-9) 600 ng/kg/min (-x-) [n=12], angiotensin-(1-9) 1,200 ng/kg/min (-▴-) [n=12], retro-inverse angiotensin-(1-9) 300 ng/kg/min (-•-) [n=12], retro-inverse angiotensin-(1-9) 600 ng/kg/min (—I—) [n=12], or retro-inverse angiotensin-(1-9) 1,200 ng/kg/min ( [symbol] ) [n=12]. Wistar Kyoto rats (WKY) were used as negative control (-♦-) [n=12] and SHR+candersartan were used as positive control (---) [n=12]. Angiotensin-(1-9) and retro-inverse angiotensin-(1-9) were administered using ALZET osmotic minipumps for 14 days. FIG. 8A: Systolic blood pressure (SBP). FIG. 8B: Diastolic blood pressure (DBP) were recorded twice a week. Data represent mean±SEM.

FIG. 9. Effect of retro-inverse angiotensin-(1-9) on β-myosin heavy chain (β-MHC) as a marker of cardiac hypertrophy in spontaneously hypertensive rats (SHR). At week 2, SHR were treated with vehicle [n=12], angiotensin-(1-9) 1,200 ng/kg/min [n=12], or retro-inverse angiotensin-(1-9) 1,200 ng/kg/min [n=12]. Wistar Kyoto rats (WKY) were used as negative control [n=12]. Angiotensin-(1-9) and retro-inverse angiotensin-(1-9) were administered using ALZET osmotic minipumps for 14 days. Animals were euthanized, hearts were obtained, homogenized and total protein were obtained by centrifugation. β-MHC was determined by Western blotting. GAPDH was used as loading control. FIG. 9A: Representative western blotting. FIG. 9B: Quantification of β-MHC levels. Data represent mean±SEM. *p<0.05, vs WKY; †p<0.05, ‡p<0.01 vs SHR+vehicle.

FIG. 10. Effect of retro-inverse angiotensin-(1-9) on area and perimeter of cardiomyocytes in spontaneously hypertensive rats (SHR). At week 2, SHR were treated with vehicle [n=12], angiotensin-(1-9) 1,200 ng/kg/min [n=12], or retro-inverse angiotensin-(1-9) 1,200 ng/kg/min [n=12]. Wistar Kyoto rats (WKY) were used as negative control [n=12]. Angiotensin-(1-9) and retro-inverse angiotensin-(1-9) were administered using ALZET osmotic minipumps for 14 days. FIG. 10A: Representative microphotographs of cross-sectional left ventricular slices stained with hematoxylin and eosin (400×). FIG. 10B: Quantification of cardiomyocyte area and FIG. 10C: perimeter in left ventricles, respectively. Results are presented as mean±SEM. *p<0.05, vs WKY; †p<0.01, ‡p<0.05 vs SHR+vehicle.

FIG. 11. Effect of retro-inverse angiotensin-(1-9) on cardiac fibrosis in spontaneously hypertensive rats (SHR). At week 2, SHR were treated with vehicle [n=12], angiotensin-(1-9) 1,200 ng/kg/min [n=12], or retro-inverse angiotensin-(1-9) 1,200 ng/kg/min [n=12]. Wistar Kyoto rats (WKY) were used as negative control [n=12]. Angiotensin-(1-9) and retro-inverse angiotensin-(1-9) were administered using ALZET osmotic minipumps for 14 days. FIG. 11A: Representative microphotographs of cross-sectional left ventricular slices stained with Picrosirius red (200×). FIG. 11B: Quantification of total collagen content in left ventricles. Results are presented as mean±SEM. *p<0.05, vs WKY; †p<0.01, ‡p<0.05 vs SHR+vehicle.

FIG. 12. Effect of retro-inverse angiotensin-(1-9) on fibroblast proliferation in the cardiac tissue from spontaneously hypertensive rats (SHR). At week 2, SHR were treated with vehicle [n=12], angiotensin-(1-9) 1,200 ng/kg/min [n=12], or retro-inverse angiotensin-(1-9) 1,200 ng/kg/min [n=12]. Wistar Kyoto rats (WKY) were used as negative control [n=12]. Angiotensin-(1-9) and retro-inverse angiotensin-(1-9) were administered using ALZET osmotic minipumps for 14 days. FIG. 12A: Representative microphotographs of transverse sections of left ventricles slices revealed using anti-Ki67, a marker of proliferation. The red arrows indicate the Ki-67 positive nuclei (200×). FIG. 12B: Quantification of Ki-67 positive nuclei per field in left ventricles slices. The number of Ki-67-positive nuclei per field was counted. A total of 20 fields around the left ventricle were counted. Results are presented as mean±SEM. *p<0.05, vs WKY; †p<0.01, ‡p<0.05 vs SHR+vehicle.

FIG. 13. Effect of retro-inverse angiotensin-(1-9) on monocyte infiltration in the cardiac tissue from spontaneously hypertensive rats (SHR). At week 2, SHR were treated with vehicle [n=12], angiotensin-(1-9) 1,200 ng/kg/min [n=12], or retro-inverse angiotensin-(1-9) 1,200 ng/kg/min [n=12]. Wistar Kyoto rats (WKY) were used as negative control [n=12]. Angiotensin-(1-9) and retro-inverse angiotensin-(1-9) were administered using ALZET osmotic minipumps for 14 days. FIG. 13A: Representative microphotographs of transverse sections of left ventricles slices revealed using anti-ED1, a marker of monocytes. The circles indicate ED1-positive cells (200×). FIG. 13B: Quantification of ED-1 positive cells per total area of the field in left ventricles slices. A total of 20 fields around the left ventricle were counted. Results are presented as mean±SEM. *p<0.05, vs WKY; †p<0.01, ‡p<0.05 vs SHR+vehicle.

FIG. 14. Effect of retro-inverse angiotensin-(1-9) on macrophage phenotypes in the cardiac tissue from spontaneously hypertensive rats (SHR). At week 2, SHR were treated with vehicle [n=12], angiotensin-(1-9) 1,200 ng/kg/min [n=12], or retro-inverse angiotensin-(1-9) 1,200 ng/kg/min [n=12]. Wistar Kyoto rats (WKY) were used as negative control [n=12]. Angiotensin-(1-9) and retro-inverse angiotensin-(1-9) were administered using ALZET osmotic minipumps for 14 days. Macrophages were isolated from hearts and analyzed by flow cytometry. M1 macrophages correspond to CD86/CD45+CD68+ cells, while M2 macrophages were identified as CD163/CD45+CD68+ cells. FIG. 14A: Quantification of M1 macrophages. FIG. 14B: Quantification of M2 macrophages. Results are presented as mean±SEM. *p<0.05, vs WKY; †p<0.01, ‡p<0.05 vs SHR+vehicle. As observed with the above cardiac fibrosis marker, collagen content assessed by picrosirius red staining, in hypertensive SHR, an increase in fibroblast with Ki-67 positive nuclei were found, confirming the occurrence of cardiac fibrosis. The treatment with retro-inverse angiotensin-(1-9) reversed the increase of fibroblast with Ki-67 positive nuclei induced by hypertension. These decreases are like those observed with angiotensin-(1-9) (FIGS. 12A and 12B). Taken together, collagen content, assessed by picrosirius red staining, and fibroblast proliferation, assessed by the presence of Ki-67 positive nuclei, indicates that retro-inverse angiotensin-(1-9) reverse cardiac fibrosis induced by hypertension, and its effect was similar to those observed with angiotensin-(1-9).

### DETAILED DESCRIPTION OF THE INVENTION

In the present invention we describe the synthesis and use of an angiotensin-(1-9) analog corresponding to a retro-inverse of angiotensin-(1-9). This invention proposes the retro-inverse strategy to give stability to the angiotensin-(1-9) peptide and thus increase its plasmatic half-life. In another aspect of this invention, it is disclosed that the retro-inverse peptide of angiotensin-(1-9) maintains the same biological activity of the native L-peptide.

The term “retro” refers to a peptide that is composed of D-amino acids in which the amino acid residues are assembled in the same sense as the native peptide. The term “inverse” refers to a peptide that is formed by L-amino acids in which the amino acid residues are assembled in the opposite direction to the native peptide. The term “retro-inverse” refers to a peptide that is composed of D-amino acids in which the amino acid residues are assembled in the opposite direction to the native peptide. Therefore, native angiotensin-(1-9) (L-amino acids, N→C direction) is Asp-Arg-Val-Tyr-Ile-His-Pro-Phe-His (SEQ ID NO: 6), i.e., DRVYIHPFH. Retro angiotensin-(1-9) (D-amino acids, N→C direction) is ^{D}D^{D}R^{D}V^{D}Y^{D}I^{D}H^{D}P^{D}F^{D}H (SEQ ID NO: 8). Inverse angiotensin-(1-9) (L-amino acids, N→C direction) is: HFPHIYVRD (SEQ ID NO: 9). The retro-inverse of angiotensin-(1-9) (D-amino acids, N→C direction) is: ° H^{D}F^{D}P^{D}H^{D}I^{D}Y^{D}V^{D}R^{D}D (SEQ ID NO: 10). The use of D-amino acids in the context of angiotensin-(1-9) derivatives modified inversely and retro-inverses is not intended to limit the use of D-amino acids in all amino acids. This invention also contemplates the use of angiotensin-(1-9) amino acid sequences where at least one of the L-amino acids was replaced by a D-amino acid.

Thus, in one embodiment, the peptide comprises or consists of the amino acid sequence HFPHIYVRD (SEQ ID NO: 9). Unless otherwise stated herein, amino acid sequences are defined in the N→C direction. The peptide comprises one or more D-amino acid residues. For instance, the peptide of SEQ ID NO: 9 may comprise at least 1, 2, 3, 4, 5, 6, 7, 8 or 9 D-amino acid residues. The remaining amino acid residues are preferably L-amino acid residues. Thus, in one embodiment, the peptide comprises the amino acid sequence ^{D}H^{D}F^{D}P^{D}H^{D}I^{D}Y^{D}V^{D}R^{D}D (SEQ ID NO: 10).

The carboxyl terminal of the peptide may be free or amidated. Thus, in one embodiment the peptide comprises or consists of the amino acid sequence NH₂-^{D}H^{D}F^{D}P^{D}H^{D}I^{D}Y^{D}V^{D}R^{D}D-COOH (SEQ ID NO: 10). In another embodiment, the peptide comprises or consists of the amino acid sequence NH₂-^{D}H^{D}F^{D}P^{D}H^{D}I^{D}Y^{D}V^{D}R^{D}D-CONH₂ (SEQ ID NO: 11). Alternative modifications of the peptide terminal sequence are also encompassed, e.g., methylation, alanine scanning, cyclization, acetylation, among others.

In another embodiment, the present invention discloses a pharmaceutical composition comprising an effective amount of the peptide, e.g., retro-inverse angiotensin-(1-9), and at least one pharmaceutically acceptable carrier, excipient, stabilizer, diluent and/or adjuvant. In a further embodiment, the present invention describes the use of said pharmaceutical composition for the preparation of medicaments useful for treating cardiovascular, renal, and cerebral diseases, for decreasing tissue remodeling, and for inducing cardioprotection.

The peptides of the present invention can be prepared by conventional methods to synthesize peptides; more specifically, using the processes described in Schroder and Lubke, The Peptides, vol. 1, published by Academic Press, New York (1966), or Izumiya et al., Synthesis of Peptides, published by Maruzen Publishing Co., Ltd., (1975), which are incorporated herein by reference. For example, an azide process, an acid chloride process, an acid anhydride process, a mixed anhydride process, a DCC process, an active ester process (for example: p-nitrophenyl ester, N-hydroxysuccinimide or cyanomethyl ester), a carbodiimidazole process, an oxidative-reducing process or a DCC/additive process can be used. The above syntheses can be carried out in a solid phase and in a liquid phase.

The peptides of the present invention are prepared in a suitable manner according to the above processes, such as are typically employed in the synthesis of peptides, generally by a step-by-step process comprising condensing an amino acid to the terminal amino acid, one by one in sequence, or by coupling peptide fragments to the terminal amino acid (amino acids side groups that are not used in the coupling reaction should be protected to avoid coupling in the wrong location).

In case a solid phase synthesis is adopted, the C-terminal amino acid is attached to an insoluble support through its carboxyl group. The insoluble carrier is not particularly limited as long as it has a binding capacity to a reactive carboxyl group. Examples of such insoluble carriers include halomethyl resins, such as chloromethyl resin or bromomethyl resin; hydroxymethyl resins, phenol resins, tert-alkyloxycarbonylhydrazide resins and the like.

An amino acid protected by an amino group is linked in sequence through the condensation of its activated carboxyl group and the reactive amino group of the previously formed peptide or chain, to be synthesized step by step. After synthesizing the entire sequence, the peptide is separated from the insoluble carrier to produce the peptide. This solid phase approach is generally described by Merrifield et al. (J Am Chem Soc. 85: 2149-2156, 1963), which is incorporated herein by reference.

The peptide can be cleaved, and the protecting groups can be removed by stirring the insoluble support or resin in anhydrous liquid HF at about 0° C. for about 20 to 90 minutes, preferably 60 minutes or bubbling HBr continuously through 1 mg/10 mL of suspension of the resin in TFA for 30 to 60 minutes at about room temperature, depending on the protective groups selected. Other methods of deprotection can also be used.

In the above process, it is preferred that the amino acids histidine, tyrosine, glutamic acid, lysine, serine, and aspartic acid are protected in the respective functional groups of the side chain. These functional groups in the side chain are protected with ordinary protecting groups that are separated after completing the reaction. The functional groups that intervene in the reaction are generally activated.

Examples of protecting groups for the hydroxy group of tyrosine include: Tos, Cl₂-Bzl, Bzl, BrZ, acetyl, benzyloxycarbonyl and the like.

Examples of protecting groups for the imino group of histidine include: trityl Bzl, Tos, benzyloxycarbonyl, and the like.

Examples of protecting groups for amino groups include: p-methoxybenzyloxycarbonyl, benzyloxycarbonyl, tert-amyloxycarbonyl, isobornyloxycarbonyl, adarnantyloxycarbonyl, trifluoroacetyl, phthalyl, Boc, Cl—Z, diphenylphosphinothioyl, formyl o-nitrophenylsulfenyl, and the like.

Examples of protecting groups for the amino group of lysine include: Tos, Boc, benzyloxycarbonyl, Cl₂-Bzl, Cl—Z and the like.

Examples of protecting groups for the serine hydroxy include: tert-butyl, Bzl and the like.

Examples of protecting groups for the carboxyl groups of glutamic acid and aspartic acid includes: the esterification of the carboxylic acids with ethanol, tert-butanol, methanol, benzyl alcohol and the like.

Examples of activated carboxyl groups include: the corresponding acid chlorides, mixed acid anhydrides, azides, acid anhydrides and active esters (esters with p-nitrophenol, pentachlorophenol, N-hydroxy 5-norbornene-2,3-dicarboxydiimide, N-hydroxybenzotriazole, N-hydroxysuccinimide, and the like).

In the above process, the PIC and pGLU residues can only be used as the amino terminus of the final peptide. In addition, the AIB residue is often coupled to the growing peptide chain in a solvent mixture that is approximately one-part DMSO to about one-part DMF.

The peptides of this invention form salts with a variety of inorganic or organic bases. Non-toxic, pharmaceutically acceptable salts are preferred, although other salts are also useful for isolating or purifying the product. Such pharmaceutically acceptable salts include metal salts, such as potassium, sodium or lithium, alkaline earth metal salts, such as magnesium or calcium, and salts derived from amino acids, such as lysine or arginine. The salts are obtained by reacting the acid form of the peptide with an equivalent of the base that supplies the desired ion in a medium in which the salt precipitates or in an aqueous medium and then lyophilized.

Similarly, the peptides form salts with a variety of inorganic and organic acids. Again, non-toxic, pharmaceutically acceptable salts are preferred, although other salts are also useful for isolating or purifying the product. Said pharmaceutically acceptable salts include those formed with sulfuric acid, hydrochloric acid, methanesulfonic acid, maleic acid, and the like. The salts are obtained by reacting the product with an equivalent amount of the acid in a medium in which the salt precipitates.

In some embodiments, the present invention describes the use of the peptide, e.g., retro-inverse angiotensin-(1-9), to prepare medicaments and/or pharmaceutical compositions for treating cardiovascular, renal, and cerebral diseases, to reduce tissue remodeling and to induce cardioprotection, especially in animals or individuals and more especially in patients who need such treatment. In another embodiments, the present invention provides, through the use of the peptide, e.g., retro-inverse angiotensin-(1-9), a method for the treatment of cardiovascular, renal, and cerebral diseases, to reduce tissue remodeling and to induce cardioprotection.

In some embodiments, the present invention provides a pharmaceutical composition comprising an effective amount of the peptide, e.g., retro-inverse angiotensin-(1-9), and at least one pharmaceutically acceptable excipient, carrier, diluent, stabilizer and/or adjuvant. The composition is preferably for the treatment of cardiovascular, renal, and cerebral diseases, to reduce tissue remodeling and to induce cardioprotection in an individual or animal in need of such treatment and comprises administering such a pharmaceutical composition to the patient. The patient can be human or animal. The use of said medicament or pharmaceutical composition seeks to elevate the plasma and/or tissue levels of retro-inverse angiotensin-(1-9), particularly to raise the levels of said peptides in the organism, particularly in the plasma, heart, kidney, brain and/or vascular bed.

In some embodiments, the medicament or pharmaceutical composition of the present invention, containing an effective amount of the peptide, e.g., retro-inverse angiotensin-(1-9), can be dispensed by all known routes of administration of medicaments described. In particular, said medicament or pharmaceutical composition can be administered by injectable and/or parenteral route (for example, and without the intention of excluding any other route, intravenous, intraarterial, intramuscular, intraperitoneal, intradermal, subcutaneous, and by direct injection to various organs, including heart, kidney and brain), by inhalation, by the use of continuous release pharmaceutical compositions, by the use of continuous release pumps, by suppositories, and orally. Said administration can be of a single dose, multiple doses, or continuous administration.

In other embodiments, pharmaceutical compositions containing the peptide, e.g. retro-inverse angiotensin-(1-9), according to the present invention can be solid or liquid, including tablets, pills, powder, wafers, dragees, capsules, coated formulations, sustained release formulations, erodible formulations, implanted devices or components derived from said apparatuses, microsphere formulations, solutions, suspensions, elixirs, aerosols and the like, containing at least one excipient, carrier, diluent, stabilizer and/or pharmaceutically acceptable adjuvant. As used herein, pharmaceutically acceptable excipients, carriers, diluents, stabilizers and/or adjuvants for the preparation of pharmaceutical compositions or medicaments of the invention are very well known in the state of the art, and can be solid, liquid or mixtures of both. As used herein, the term “liquid carriers” refers to diluents and/or excipients including water, saline, dextrose solution and glycol solution, especially when the parenteral and/or injection route is used as the route of administration. As used herein, the term “carrier and/or diluent” can also be an oil, such as for example those derived from petroleum, oils of animal and/or vegetable origin or synthetic oils. Examples of preferred oils in the invention include peanut oil, soybean oil, mineral oil, sesame oil, corn oil, marigold oil, among others. As used herein, the term “excipients” refers starch, cellulose, talc, glucose, lactose, sucrose, gelatin, malt, rice, flour, chalk, silica gel, magnesium stearate, sodium stearate, glycerol monostearate, sodium chloride, dehydrated skim milk, glycerol, propylene glycol, water, ethanol, among others. Other transporters, diluents, stabilizers, excipients and/or adjuvants, which are not named here, are obvious to an expert in the state of the art.

In some embodiments, the pharmaceutical compositions may be in the form of a sterile injectable preparation, for example as a sterile injectable aqueous or oleaginous suspension. This suspension can be formulated according to the known art using suitable dispersing or wetting agents and suspending agents. The sterile injectable preparation can also be a sterile injectable solution or suspension in a non-toxic and parenterally acceptable diluent or solvent, for example as a solution in 1,3-butanediol. Among the acceptable vehicles and solvents that may be employed are water, Ringer's solution, and isotonic sodium chloride solution. In addition, sterile fixed oils are conventionally employed as a solvent or suspending medium. For this purpose, any insipid, fixed oil may be employed, including mono or synthetic diglycerides. In addition, fatty acids such as oleic acid find use in the preparation of injectables.

In yet another embodiment, the peptide, e.g., retro-inverse angiotensin-(1-9), can also be administered in the form of suppositories for rectal administration of the drug. These compositions can be prepared by mixing the medicament with a suitable non-irritating excipient that is solid at normal temperatures but liquid at the rectal temperature and, therefore, will melt in the rectum to release the medicament. Such materials are cocoa butter and polyethylene glycols.

In other embodiments, the pharmaceutical composition or medicament of the present invention may be subject to conventional pharmaceutical processes, such as sterilization, and may contain other conventional pharmaceutical additives such as preservatives, stabilizers, emulsifying agents, wetting agents, salts for adjusting osmotic pressure, or buffers. To regulate the pH, among others. Transporters, stabilizers, diluents, excipients and/or adjuvants and their formulations can be found in Martin, “Remington's Pharmaceutical Sciences”, 15 “Ed.; Mack Publishing Co., Easton (1975), see for example pages 1405-1412 and 1461-1487.

In another embodiments, pharmaceutical compositions containing the peptide, e.g., retro-inverse angiotensin-(1-9), generally contain an effective amount of the active compound together with a suitable amount of one or more carriers, stabilizers, diluents, excipients and/or adjuvants, in such a manner as to make it possible to prepare the dose and form suitable for the proper administration of the peptide, e.g., retro-inverse angiotensin-(1-9), to the patient. In the practice of the methods of treatment of the invention, the particular dosage of a pharmaceutical composition or medicament to be administered to the subject will depend on many variables including the state of the disease, the severity of the disease, the administration scheme, the age, the physical characteristics of the subject, etc. Appropriate doses can be established using clinical approaches as understood by those in the field. The term “effective amount” refers to the dose and the period of time necessary to achieve the necessary therapeutic result, that is, to decrease tissue remodeling, increase cardioprotection, and to effectively treat cardiovascular, renal, and cerebral diseases. The effective amount may depend on many factors, such as the state of advance of the disease, age, sex, weight of the individual, the presence of other diseases, of the intake of other medications simultaneously, of race, among other things.

In some embodiments of the present invention, chemically synthesized retro-inverse angiotensin-(1-9) is used. In some embodiments, rats and mice are used as an example of mammals to which the method of treatment can be applied and to test the use of the peptide, e.g., retro-inverse angiotensin-(1-9), in the form of a medicament and/or pharmaceutical composition. Animal models, including small mammals such as rat and mice, to study cardiovascular, renal, and cerebral disease, tissue remodeling and cardioprotection, are very well accepted in the state of the art (Everette et al., Hypertension 23: 587-93, 1994; Indolfi et al., Circulation, 92: 1230-5, 1995). Although the use of the peptide, e.g., retro-inverse angiotensin-(1-9), and/or pharmaceutical compositions containing it and thereof are exemplified in rats and mice, it is understood that the present invention is extends to any mammal, for example, and without limitation to human, mouse, rabbits, primates, dogs, cats, pets in general, farm animals, etc. The use of the rat and mice models also does not exclude its use in humans that requires such treatment.

Herein the term “cardiovascular disease” refers to any cardiovascular disease or disorder known in the art, including, but not limited to, heart failure (congestive heart failure, compensated heart failure, decompensated heart failure, and the like), restenosis, hypertension (low-renin hypertension; salt-sensitive hypertension; low-renin, salt-sensitive hypertension; primary pulmonary hypertension; thromboembolic pulmonary hypertension; pregnancy-induced hypertension; renovascular hypertension), heart hypertrophy, diastolic dysfunction, coronary artery disease, myocardial infarctions, cerebral infarctions, atherosclerosis, atherogenesis, cerebrovascular disease, angina, (including chronic, stable, unstable and variant (Prinzmetal) angina pectoris), aneurysm, ischemic heart disease, cerebral ischemia, myocardial ischemia, thrombosis, platelet aggregation, platelet adhesion, smooth muscle cell proliferation, vascular or non-vascular complications associated with the use of medical devices, wounds associated with the use of medical devices, vascular or non-vascular wall damage, peripheral vascular disease, neointimal hyperplasia following percutaneous transluminal coronary angiograph, vascular grafting, coronary artery bypass surgery, thromboembolic events, post-angioplasty restenosis, coronary plaque inflammation, hypercholesterolemia, embolism, stroke, shock, arrhythmia, atrial fibrillation or atrial flutter, thrombotic occlusion and reclusion cerebrovascular incidents, left ventricular dysfunction and hypertrophy, and the like. An embodiment of this invention involves the administration of the peptide, e.g., retro-inverse angiotensin-(1-9), to a patient with any of the above-described cardiovascular disease. In another embodiment of this invention, it is also considered that the peptide, e.g., retro-inverse angiotensin-(1-9), can be administered to subjects together o in a coadministration with a well-known cardiovascular drug. For example, the peptide, e.g., retro-inverse angiotensin-(1-9), can be co-administered angiotensin I converting enzyme inhibitors, with angiotensin II receptor antagonist, with Rho kinase inhibitors, with diuretics, with calcium channel inhibitors, with renin inhibitors, with other retro-inverse peptides, and with other cardiovascular drugs thereof. Herein, angiotensin I converting enzyme inhibitors include, but they are not limited to, lisinopril, enalapril, captopril, zofenopril, ramipril, quinapril, perindopril, benazepril and fosinopril. Herein, angiotensin II receptor antagonists include, but they are not limited to, valsartan, telmisartan, losartan, irbesartan, olmesartan, candesartan, eprosartan and saralasina. Herein, calcium channel inhibitors include, but they are not limited to, dihydropyridines (nicardipine, nifedipine, amlodipine, felodipine, nitrendipine, nisoldipine, isradipine, nimodipine), benzothiazepines (diltiazem, clentiazem) and phenylalkylamines (verapamil, galopamil, anipamil, RO5967, falipamil). Herein, Rho kinase inhibitors include, but they are not limited to, fasudil, hidroxifasudil, 3-(4-pyridil)-1H-indol, (S) (+) 2 methyl 1 [(4 methyl-5-isoquinolinyl)sulphonyl]homopyperazine, N (4 pyridyl) N′ (2,4,6-trichlorophenyl) urea. Herein, diuretics include, but they are not limited to, thiazide diuretics (bendroflumethiazide, benzythiazide, chlorothiazide, chlortalidone, hydrochlorothiazide, hydroflumethiazide, indapamide, methyclothiazide, metolazone, polythiazide, quinetazone, trichlormethiazide, xipamide), loop diuretics (furosemide, torasemide, bumetanide, etacrynic acid), carbonic anhydrase inhibitors (acetazolamide, dorzolamide), sodium channel inhibitors (amiloride, triamterene), aldosterone antagonists (spironolactone, canrenoate, eplerenone) and osmotic compounds (mannitol). Herein, renin inhibitors include, but they are not limited to, pepstatin, CGP2928, remikiren, enalkiren, zankiren, aliskiren. Herein, other retro-inverse peptides include, but they are not limited to, retro-inverse bradykinin and retro-inverse AT1R.

As used herein, the method to reduce tissue remodeling comprises the reversing, inhibiting and/or decreasing cardiovascular (heart and blood vessels), pulmonary, renal and/or cerebral remodeling in an individual or an animal. The term “remodeling” refers as the complex change suffered by the organs when subjected to stress conditions. The organs that are of particular interest in this invention to prevent remodeling by the use of retro-inverse angiotensin-(1-9) correspond to the heart, to the blood vessels, to the kidney, and to the brain, without excluding other organs that could undergo remodeling and that could be treated with retro-inverse angiotensin-(1-9). The remodeling should be understood in its broadest possible form and involve numerous cellular, biochemical and/or physiological processes, which comprise one or all of the processes selected between cardiomyocyte hypertrophy, neointima formation, restenosis, fibroblast hyperplasia, hyperplasia of smooth muscle cells, fibrosis, collagen deposition, inflammation, apoptosis, necrosis and/or autophagy, oxidation. The hypertrophy of the cardiomyocytes corresponds to the enlargement of the cardiomyocytes, with an increase in the content of intracellular proteins, especially those associated with the contractile machinery, and with the re-expression of fetal proteins such as the heavy chain of β-myosin (β-MHC) and the atrial natriuretic factor (ANF). The hypertrophy of cardiomyocytes is associated with cardiac hypertrophy. Cardiac hypertrophy corresponds to the growth observed in the heart in athletes of high competition (physiological or benign hypertrophy) or in people with hypertension or after a myocardial infarction (pathological hypertrophy). Formation of the neointima corresponds to the formation of undifferentiated new tissue or of various types in the blood vessels due to damage or for any other cause, including restenosis. Restenosis is the re-narrowing of any blood vessel, for example re-narrowing of a coronary artery after angioplasty. Restenosis can be caused by many other pathologies and causes. Hyperplasia of fibroblasts corresponds to an increase in the number of fibroblasts due to an increase in proliferation. Smooth muscle cell hyperplasia corresponds to an increase in the number of smooth muscle cells due to an increase in proliferation. The term “fibrosis” refers to the increase in the content of extracellular matrix in a tissue by accumulation of proteins such as collagen, fibronectin, elastin, among others. Fibrosis also involves the proliferation of fibroblast and their differentiation to myofibroblast. Oxidative stress is understood as an increase in reactive oxygen species and is caused by an imbalance between the synthesis and degradation of reactive oxygen species. The reactive oxygen species correspond to superoxide anion (O₂⁻), hydrogen peroxide (H₂O₂), hydroxyl radical (OH•) and/or to products of these species with other molecules generating for example peroxynitrite (NOO•). The synthesis of reactive oxygen species can be synthesized in the oxygen transport chain of the mitochondria, NADPH oxidase, xanthine oxidase, NO synthase, by inorganic reactions such as the Fenton reaction and the Haber-Fenton reaction, among others. The degradation mechanisms of the reactive oxygen species include natural antioxidants (for example vitamin C, alpha tocopherol, uric acid, mannitol) and enzymatic systems (for example superoxide dismutase, catalase, glutathione peroxidase, among others).

Herein the term “cardioprotection” refers to all phenomena, processes or mechanisms involved in the reduction of damage or reduction of cardiomyocyte death. Apoptosis, necrosis, and autophagy correspond to different types of cell death. In this invention, the terms hypertrophy, neointima formation, restenosis, hyperplasia, cardioprotection, fibrosis, collagen deposition, inflammation, oxidative stress, apoptosis, necrosis, and autophagy should be understood in their broadest context.

Accordingly, in further aspects the present invention provides a peptide (e.g., retro-inverse angiotensin-(1-9)) or pharmaceutical composition as defined above, for use in:
(i) treating a cardiovascular disease, e.g., a cardiovascular disease as defined above;
(ii) preventing, reverting, inhibiting, and/or reducing cardiovascular, pulmonary, renal, and/or cerebral remodeling, preferably wherein cardiovascular remodeling comprises cardiac fibrosis, cardiomyocyte hypertrophy, cardiovascular inflammation, and/or fibroblast proliferation;
(iii) inducing cardioprotection, preferably decreasing myocardial infarct size;

The following examples are illustrative of the present invention but should not be construed as limitations thereof.

### EXAMPLES

### Example 1. Retro-Inverse Angiotensin-(1-9) Peptide Synthesis

Angiotensin-(1-9) (SEQ ID NO: 6) and retro-inverse angiotensin-(1-9) (SEQ ID NO: 10) and retro-inverse angiotensin-(1-9) amide (^{D}H^{D}F^{D}P^{D}H^{D}I^{D}Y^{D}V^{D}R^{D}D-CONH₂, SEQ ID NO: 11) were synthesized in the Curauma Biotechnology Nucleus (NBC) of the Pontificia Universidad Católica de Valparaiso, using the procedure described herein.

A desired volume of solvent was taken and added to a tube with alumina. Then, in another tube, the sieve was activated by treating at 50° C. for 15 min. Finally, the molecular sieve was dried and the solvent from the tube with alumina was added. All the solvents used were dried with alumina and molecular sieve.

Resin treatment: The amount of resin needed was weighed in a reactor and washed it twice with dichloromethane (DCM) after draining the reactor and adding DCM, leaving 10 min for swelling. Then the reactor was drained.

Coupling of the first amino acid: 1.6 mmol of the Fmoc-amino acid were weighed for each gram of resin and dissolved in the minimum possible DCM. 3 equivalents (relative to the amino acid) of diisopropylethylamine (DIEA) were added, stirred for 15 min, another 2 equivalents of DIEA were added and stirred for 1-2 h. Then 0.5 mL of methanol per gram of resin was added and stirred for 5 min. Subsequently, it was washed 3 times with DCM, 2 times with dimethylformamide (DMF) and 2 more times with DCM and the resin was dried.

Determination of substitution: 5-10 mg of dry resin-amino acid were weighed and 1 mL of piperidine 20% (without Triton X-100) was added. It was stirred for 20 min and 30 μL of this solution was taken and diluted with 3 mL DMF in a quartz cell. The absorbance of the samples at 290-300 nm was then measured in a spectrophotometer (using DMF as blank) and the degree of substitution was calculated with the formula (101×A)/(7.8×w) where A=Absorbance and w=mg of resin.

Synthesis from the second amino acid: The resin was washed with DMF. The Fmoc was removed with 20% piperidine/DMF and washed with DMF and then with DMF and DCM. The Kaiser test was performed (with a resin peptide sample). Subsequently, it was washed with DMF. Peptide cleavage was performed by acidolysis with trifluoroacetic acid (TFA), using triethylsilane and water as scavengers (94:3:3, v/v/v) for 60-90 min. The TFA was removed with a stream of N₂ and the oily residue was precipitated with dry tert-butyl ether. The crude peptide was recovered by centrifugation and decantation of the tert-butyl ether phase. The amide group in the carboxyl end of the peptide con be maintained (retro-inverse angiotensin-(1-9) amide) or removed by hydrolysis (retro-inverse angiotensin-(1-9)).

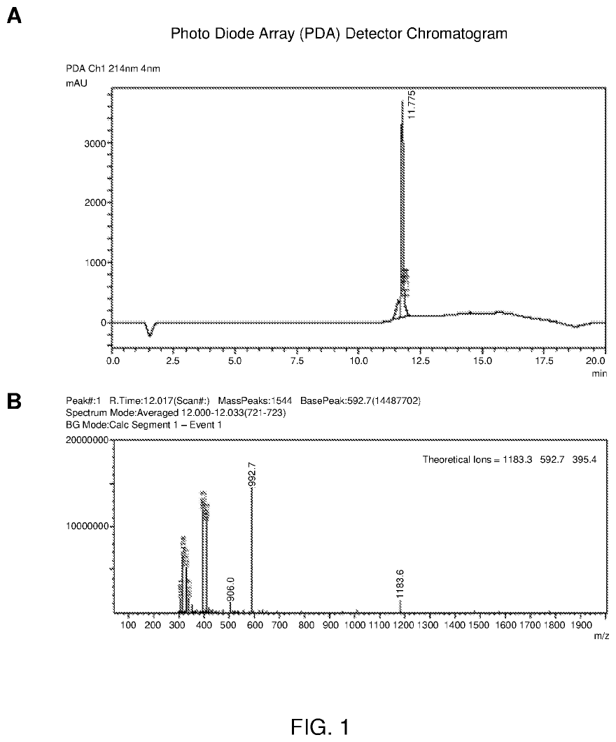

Purity and identity of peptides: Purity control was performed with HPLC coupled to a photo diode array detector. With this method a purity >98% was obtained (FIG. 1A). Identity of the peptide was verified by mass spectrometry analysis (FIG. 1B). The same characterization was used in the retro-inverse angiotensin-(1-9) amide.

### Example 2. Stability of Retro-Inverse Angiotensin-(1-9) in Human Serum

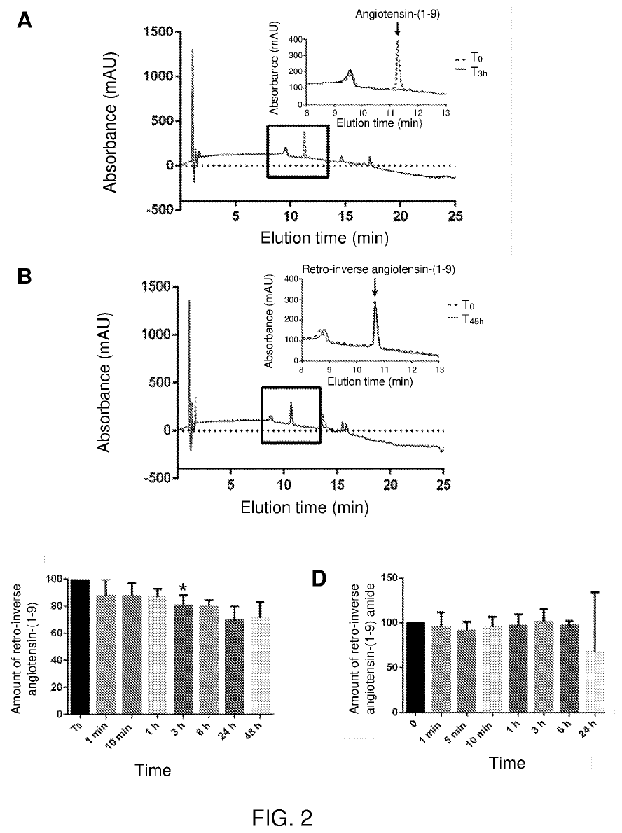

The retro-inverse peptides were dissolved in PBS to obtain a final concentration of 1 mM. From this stock, an aliquot of 50 μL was taken and diluted to 450 μL of human serum (Sigma Aldrich from human male AB plasma, USA origin, sterile-filtered) at 37° C. under agitation, to obtain an initial concentration of 20 μM and incubated for 48 h. Aliquots of 50 μL were extracted at 0, 5, 10 min and 1, 3, 6, 24 and 48 h and added to 200 μL of methanol at 4° C. for 30 min to precipitate serum proteins. The samples were then centrifuged at 13,000 g in Hermle Cooling Centrifuge Z 326 K 311.00 V01 at 4° C. for 30 min. The supernatants (approximately 200 μL) were obtained, placed in vials, and then analyzed by HPLC. All analyzes were performed on the Perkin Elmer auto sampler flexo HPLC, using a reverse phase 4.6×100 mm column with a particle size of 3.5 μM (Waters XBridge Peptide BEH C18 Column). Mobile phase A was 100% acetonitrile and phase B was TFA 0.0004% in Milli-Q water. The initial composition of eluent was 5% A and 95% B for 1 min and then a linear gradient to reach 100% A in 19 min and then return to the initial composition in 4 min, with a total run time of 25 min. The eluent flow was 0.9 mL/min and the temperature of the column was adjusted to 26° C. L measurement was performed at a UV signal of 220 nm wavelength. Where 50 μL was injected per sample. Peptides were identified based on their retention time in the respective chromatogram obtained in the Chromera-HPLC flexar program. Angiotensin-(1-9) used as control, was completely degraded after 3 h of incubation with human serum (FIG. 2A). Retro-inverse angiotensin-(1-9) showed <20% degradation after 48 h incubation with human serum (FIGS. 2B and 2C)). Retro-inverse angiotensin-(1-9) amide showed no significant degradation after 24 h incubation with human serum (FIG. 2D)).

### Example 3. Effect of Retro-Inverse Angiotensin-(1-9) on Cardiomyocyte Hypertrophy

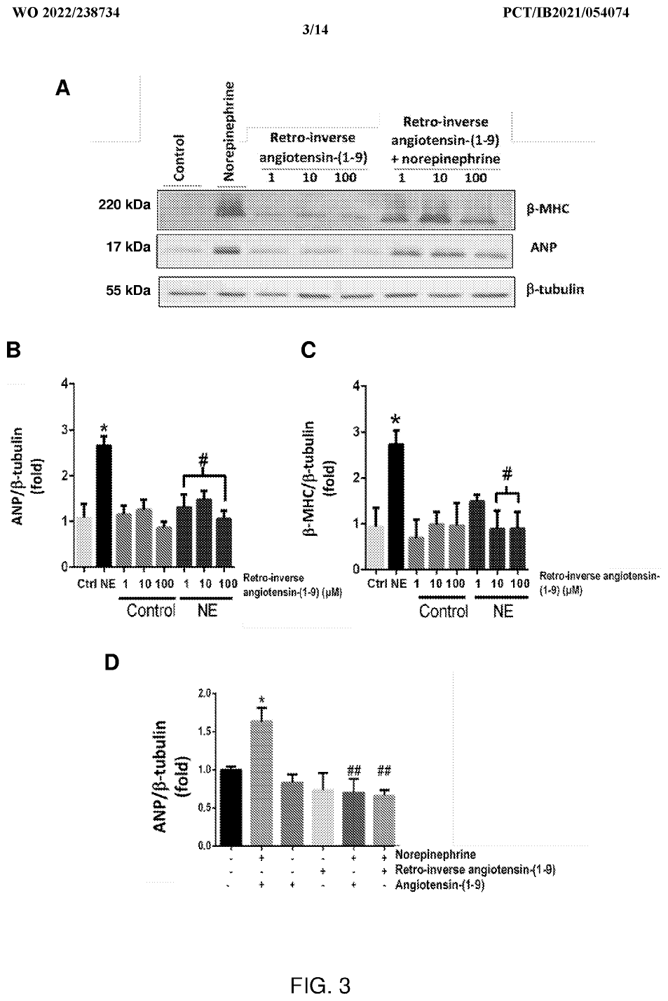

Cardiac myocytes were isolated from neonatal Sprague-Dawley rat ventricles. Cardiac myocytes were plated at 70% final density in gelatin-coated wells (12-well plates) or in 60-mm Petri dishes and maintained at 37° C. in a humidified atmosphere of 5% CO₂/95% air for 24 h in Dulbecco's Modified Eagle's medium [(DMEM)/M199](4:1) containing 10% fetal bovine serum and 5% fetal calf serum. Serum was withdrawn 24 h before preincubation with 1, 10 or 100 μM retro-inverse angiotensin-(1-9) for 1 h; then, 10 μM norepinephrine (Sigma, St Louis, Missouri, USA) were added and the cultures were incubated for 24 h. Then cells were washed with PBS at 4° C. and then cells were lysed with 50 μL of RIPA lysis buffer (Tris-HCl 10 mM pH 7.4, EDTA 5 mM, NaCl 50 mM, deoxycholic acid 1%, Triton X-100 1% v/v) supplemented with phosphatase and protease inhibitors (Roche). After centrifugation at 12,000 r.p.m. for 15 min at 4° C., the supernatat was recovered; total protein content was determined by using Bradford (BioRad protein assay, BioRad, Hercules, CA, USA). Proteins were denatured in SDS-PAGE 4× buffer and resolved in 12% PAGE-SDS. After electrotransference, beta-myosin heavy chain (β-MHC) and atrial natriuretic peptide (ANP) were used as hypertrophic markers. β-tubulin was used as loading control. As expected, norepinephrine 10 μM for 48 h, a cardiomyocyte hypertrophy inducer, triggered β-MHC and ANP increase (FIG. 3). Treatment with retro-inverse angiotensin-(1-9) inhibited, in a dose response manner, the increase of both β-MHC and ANP induced by norepinephrine (FIGS. 3A, 3B, and 3C). Retro-inverse angiotensin-(1-9) amide also blocked the increase of ANP induced by norepinephrine (FIG. 3D).

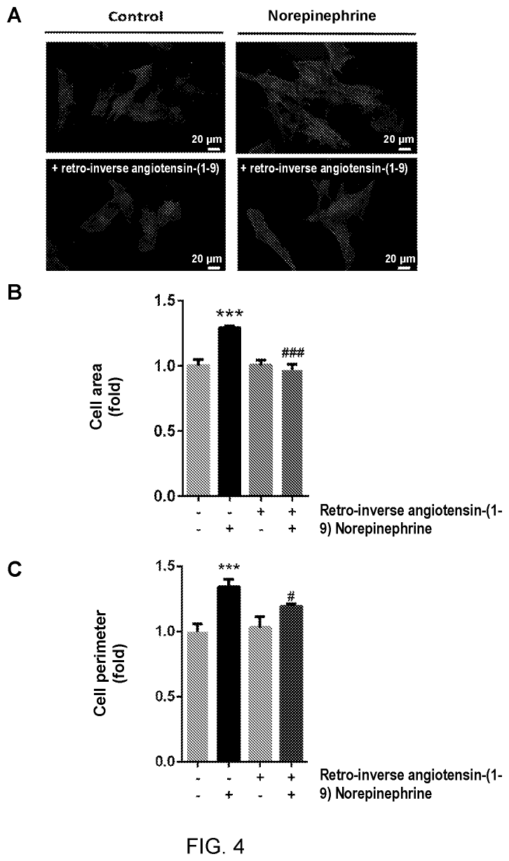

Cardiomyocyte hypertrophy is characterized by an increase of cell area and perimeter. Cell area and perimeter were analyzed as follows. Cells were grown on coverslips, stimulated and fixed, incubating with cytoskeleton stability buffer [10 mM MES pH 6.0, 150 mM NaCl, 5 mM EDTA, 3% sucrose, 5 mM MgCl₂] for 5 min. Cell area and perimeter were determined in cells fixed with 4% paraformaldehyde for 10 min and permeabilized with 0.2% Triton X100 for 6 min. Non-specific sites were blocked with 3% bovine serum albumin (BSA) in phosphate buffered saline (PBS) for 1 h. Cells were washed with PBS and incubated with phalloidin-rhodamine 1:500 (to stain F-actin, red) at room temperature for 45 min. Hoechst (1:1000) was used to stain the nuclei (blue). Coverslips were mounted in DakoCytomation Fluorescent Mounting Medium. Cell staining was evaluated by epifluorescence microscopy. At least 100 cells from randomly selected fields were analyzed using Image J software (NIH). Norepinephrine 10 μM for 48 h increased cardiomyocyte area and perimeter (FIG. 4). Treatment with retro-inverse angiotensin-(1-9) 100 μM inhibited, in a dose response manner, the increase of both cell area (FIGS. 4A and 4B) and cell perimeter (FIGS. 4A and 4C) induced by norepinephrine.

### Example 4. Effect of Retro-Inverse Angiotensin-(1-9) on Fibrosis

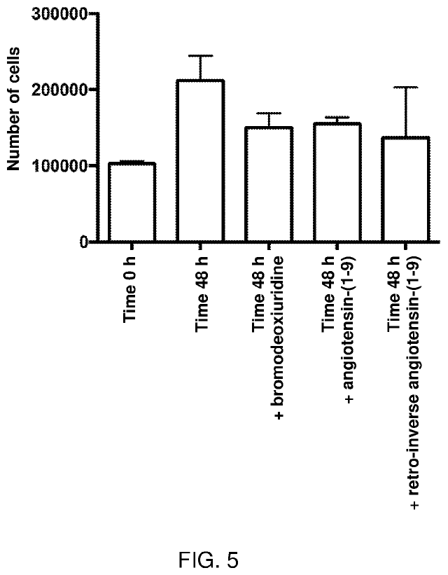

Male Sprague-Dawley rats (250-300 g) were anesthetized with ketamine-xylazine (66 and 1.6 mg/kg i.p., respectively). Adult rat cardiac fibroblasts were isolated by retrograde aortic perfusion. Briefly, hearts were digested with collagenase type B solution for 1 h, and cells were centrifuged at 500 rpm for 2 min. The supernatant, mainly adult rat cardiac fibroblasts, was centrifuged at 1000 rpm for 10 min, resuspended in Dulbecco's Modified Eagle Medium: Nutrient Mixture F-12 (DMEM F-12) plus 15% fetal bovine serum (FBS) and then seeded in nontreated culture dishes during 3 h. Cells were washed with phosphate buffered saline to eliminate debris and nonadherent cells. Adult rat cardiac fibroblasts were used at passage one and seeded on 60 mm cell culture dishes in DMEM F-12 plus 10% FBS and incubated at 378° C. for 3 h. To synchronize cells, they were washed and incubated in DMEM F-12 (without serum, 0% FBS) for 24 h before experimentation. Cell proliferation was determined in cardiac fibroblasts incubated for 24 h with and without retro-inverse angiotensin-(1-9) in DMEM F-12 5% FBS. Cells were trypsinized and cell number was determined using trypan blue method in a Neubauer chamber. Retro-inverse angiotensin-(1-9) inhibited fibroblast proliferation in the same extent that angiotensin-(1-9) does. Inhibition of fibroblast proliferation by retro-inverse angiotensin-(1-9) was also comparable to bromodeoxyuridine (FIG. 5).

### Example 5. Effect of Retro-Inverse Angiotensin-(1-9) on Cardioprotection

Neonatal rat ventricular myocytes (NRVM) were isolated from one- to three-day-old Sprague Dawley rats. Cells were pre-plated to discard non-myocyte cells and the myocyte-enriched fraction was plated on gelatin-precoated 35 mm dishes and grown in DMEM/M199 (4:1) medium with 10% (w/v) fetal bovine serum (FBS) and 100 mM bromodeoxyuridine for 24 h before the experiments. Ischemia was induced by incubating the cardiomyocytes in ischemia-mimicking solution (ischemic medium) containing HEPES 5 mM, 2-deoxy-D-glucose 10 mM, NaCl 139 mM, KCl 12 mM, MgCl₂ 0.5 mM, CaCl₂ 1.3 mM and lactic acid 20 mM, pH 6.2, under O₂<1%, 5% CO₂ and 95% nitrogen at 37° C. for 8 h. Subsequently for simulated reperfusion, ischemia-mimicking solution was replaced by DMEM/M199 (4:1) containing 10% (w/v) FBS and NRVM were incubated for 16 h in 95% air and 5% CO₂. Parallel NRVM were assigned to a control group which was exposed to normoxic conditions in a control medium containing (in mM) 5 HEPES 5 mM, D-glucose 23 mM, NaCl 139 mM, KCl 4.7 mM, MgCl₂ 0.5 mM, CaCl₂ 1.3 mM, pH 7.4 under 95% air and 5% CO₂ for 8 h. Then, control medium was replaced with DMEM/M199 (4:1) containing 10% (w/v) FBS under 95% air and 5% CO₂ for 16 h.

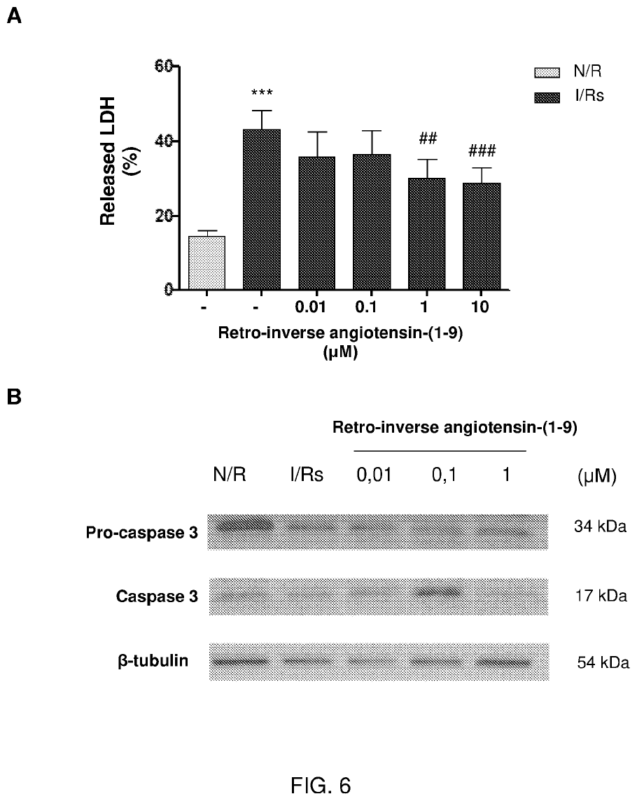

For the evaluation of necrosis, the activity of lactate dehydrogenase (LDH) was measured spectrophotometrically at 490 nm in samples of the culture medium after normoxia (control) and sl/R both at 8 h and 16 h, using the kit CytoTox 96® Non-Radioactive Cytotoxicity Assay, Promega (Corp., Madison, WI, USA), according to the manufacturer's instructions. Apoptosis was evaluated by measuring procaspase 3 cleavage to caspase 3 by western blotting. Retro-inverse angiotensin-(1-9) inhibited in a dose response manner the cardiomyocyte necrosis induced by ischemia reperfusion. Significative necrosis inhibition was obtained with 1 and 10 μM retro-inverse angiotensin-(1-9) (FIG. 6A). Retro-inverse angiotensin-(1-9) 10 μM also inhibited cardiomyocyte apoptosis induced by ischemia reperfusion, as determined by inhibition of procaspase 3 cleavage (FIG. 6B).

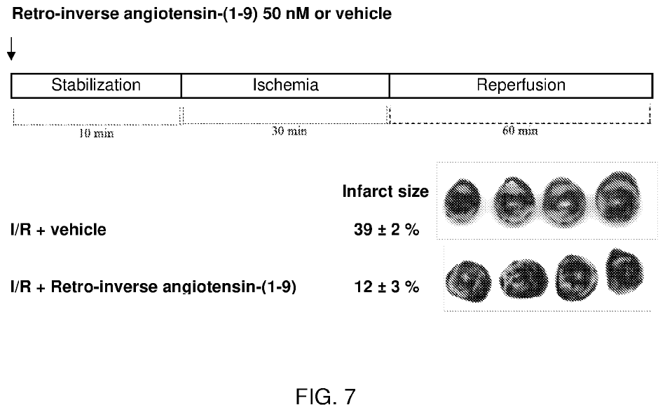

The cardioprotective effects of retro-inverse angiotensin-(1-9) were also investigated in isolated hearts from adult male Sprague-Dawley rats (250-300 g) subjected to ischemia/reperfusion (I/R) using a Langendorff procedure. Rats were anesthetized with pentobarbital [80 mg/kg intraperitoneally (i.p.)] and heparin 100 U/kg was injected into the right atria. Hearts were rapidly harvested and perfused through the aorta with Krebs-Henseleit solution containing NaCl 128.3 mM, KCl 4.7 mM, CaCl₂ 1.35 mM, MgSO₄ 1.1 mM, NaHCO₃ 20.2 mM, NaH₂PO₄ 0.4 mM and glucose 11.1 mM, pH 7.4 (equilibrated with a gas mixture of 95% O₂ and 5% CO₂ at 37° C.), using a peristaltic pump (Gilson Miniplus 3, France). A latex balloon connected to a pressure transducer was placed through the left atrium and mitral valve into the left ventricle. The balloon was filled with saline to determine isovolumetric intraventricular pressure. Perfusion flow was 10-14 mL/min. Hearts were placed in a heated chamber and paced at 240-300 beats/min with platinum electrodes, using a Grass stimulator (pulses of 5V, 1 ms). In order to assess the effect of retro-inverse angiotensin-(1-9) in the infarct size in the ex vivo model of I/R injury, adult rat hearts were stabilized for 10 min, followed by 30 min of global ischemia and reperfusion with 50 nM retro-inverse angiotensin-(1-9) for 60 min. The infarct size was determined using 2,3,5-triphenyltetrazolium chloride (TTC) staining. At the end of reperfusion, hearts were first perfused with 1% TTC solution at 37° C. for 15 min and then frozen at −20° C. for 1 h. Then, hearts were cut into six slices and stored with formaldehyde 10% for 48 h before measuring the percentage of infarct size using the software image J (NIH, Boston, MA, USA http://rsb.info.nih.gov/ij). Treatment with retro-inverse angiotensin-(1-9) decrease around 70% the infarct size (FIG. 7).

### Example 6. Effect of Retro-Inverse Angiotensin-(1-9) on Hypertension and Hypertension-Dependent Heart Damage

Experimental animal model. All procedures were performed in accordance with the Guide for the Care and Use of Laboratory Animals published by the U.S. National Institutes of Health (NIH Publication 85 23, 1985) and approved by our Institutional Bioethics Committee. The animals were housed under the continuous professional monitoring of the Medical Veterinary staff in the animal facility of the Pontificia Universidad Católica de Chile, under the following conditions: 12-h light-dark cycle; 21° C. ambient temperature; 50% humidity; adequate ventilation; noise-free environment; and food and water ad libitum. Male spontaneously hypertensive rats (SHR, age 12 weeks) and normotensive Wistar Kyoto rats (WKY, 12 weeks age) were used. SHR with arterial pressure >140 mm Hg were randomized into nine experimental groups: i) SHR+vehicle (n=12), ii) SHR+angiotensin-(1-9) (300 ng/kg/min) (n=12), iii) SHR+angiotensin-(1-9) (600 ng/kg/min) (n=12), iv) SHR+angiotensin-(1-9) (1200 ng/kg/min) (n=12), v) SHR+retro-inverse angiotensin-(1-9) (300 ng/kg/min) (n=12), vi) SHR+retro-inverse angiotensin-(1-9) (600 ng/kg/min) (n=12), vii) SHR+retro-inverse angiotensin-(1-9) (1200 ng/kg/min) (n=12), viii) Untreated WKY rats were used as normotensive negative control, ix) SHR+candersartan were used as a positive control of an effective hypotensive treatment. Angiotensin-(1-9) and retro-inverse angiotensin-(1-9) were administered with ALZET 2002 osmotic minipumps, using a pumping rate of 0.5 mL/h during 14 days (Alzet, Cupertino, CA, USA). These osmotic minipumps were implanted in the jugular vein while the rats were sedated with ketamine HCl and xylazine (35 and 7 mg/kg, respectively by intraperitoneal injection). All groups completed two weeks of treatment. Systolic blood pressure (SBP), diastolic blood pressure (DBP) and body weight (BW) were measured at the end of the treatment period and the animals were then sacrificed.

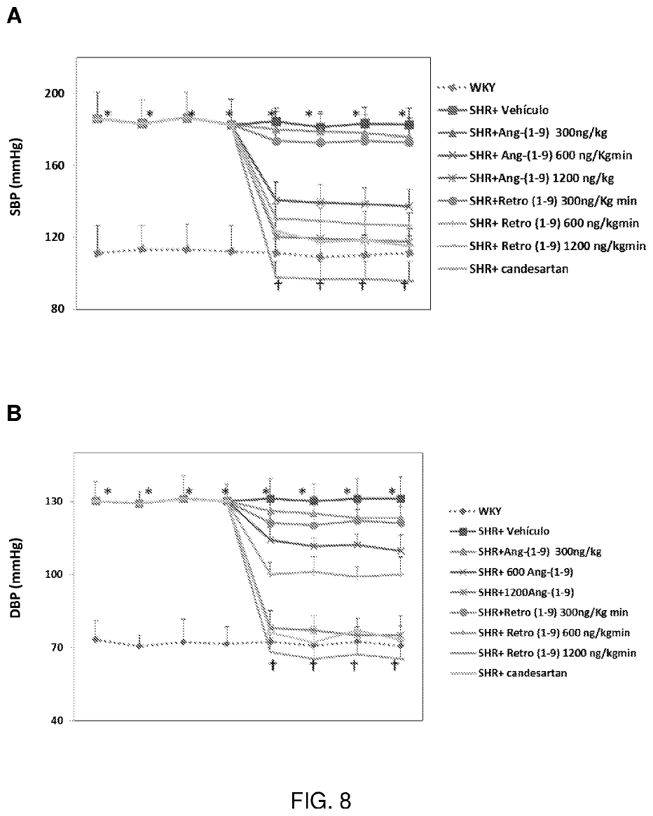

Hemodynamic evaluations. SBP and DBP were measured twice per week by a researcher blinded to study group, using a CODA 2 noninvasive pressure device with volume-pressure recording (Kent Scientific Corporation, Torrington, CT, USA). Measurements were obtained in conscious rats restrained in a thermal plastic chamber, as previously described (Libby P et al Circulation. 2002; 105:1135-43). Data show that retro-inverse angiotensin-(1-9) reduced SBP and DBP in a dose-dependent manner. This reduction is similar to those observed with angiotensin-(1-9) (FIGS. 8A and 8B).

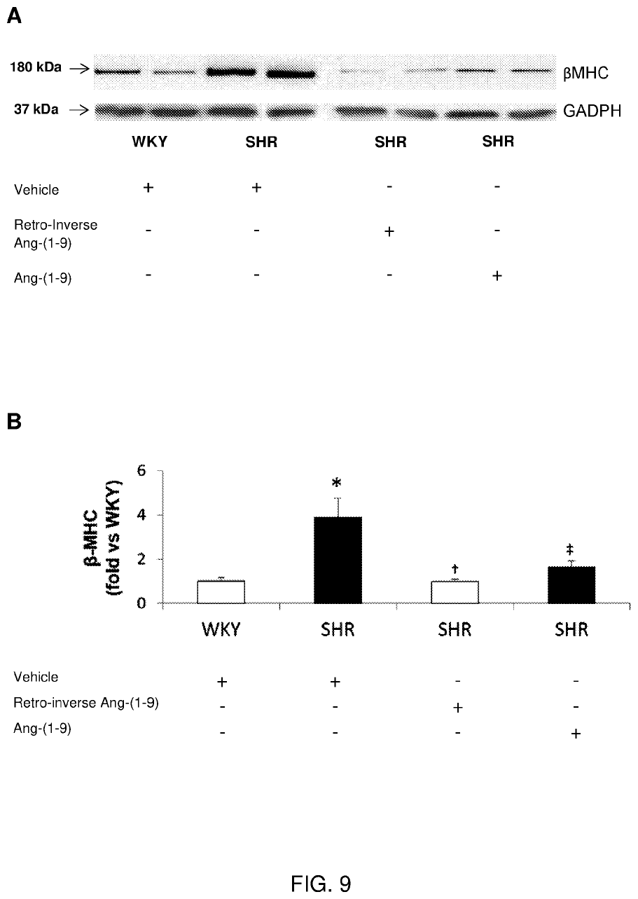

Measurement of β-MHC protein level to assess cardiac hypertrophy. β-MHC protein levels were determined by Western blotting. Left ventricles were frozen in liquid nitrogen and stored at −80° C. until processing. The tissues were homogenized and lysed with lysis buffer with low concentrations of detergent (50 mM HEPES, 150 mM NaCl, 2 mM MgCl₂, 1 mM EGTA, 1% Triton X-100, and 10% glycerol) supplemented with protease inhibitors (2 μg/mL aprotinin, 10 μg/mL leupeptin, and 1 mM PMSF) and phosphatase inhibitors (4.5 mg/mL NaP₂O₇, 10 mM NaF, and 1 mM Na₃VO4) on ice. Equal amounts of protein (25 μg) were loaded and resolved on a 10% SDS-PAGE gel and transferred to a nitrocellulose membrane (Bio Rad). After blocking with 7% non-fat milk (for non-phosphorylated proteins) or BSA 5% (for phosphorylated proteins) for 1 h at room temperature, the blots were incubated overnight at 4° C. with anti-β-MHC (rabbit polyclonal: 1/1000, Calbiochem), Blots were then washed and incubated with a secondary antibody, HRP-conjugated goat anti-rabbit IgG (1:5,000, Thermo Scientific) for 2 h. The relative amount of protein was estimated by chemiluminescence using the ECL plus kit (Perkin Elmer), which contains the substrate for HRP. Digital images obtained from the photographic films were analyzed by densitometry using Image J software (NIH, USA). A GAPDH mouse monoclonal antibody (1:1000) (Santa Cruz Biotechnology Inc) was used as protein loading control. In hypertensive SHR, an increase in β-MHC protein levels was observed, indicating the occurrence of cardiac hypertrophy. The treatment with retro-inverse angiotensin-(1-9) reversed the increase in β-MHC induced by hypertension. This decrease is like those observed with angiotensin-(1-9) (FIGS. 9A and 9B).

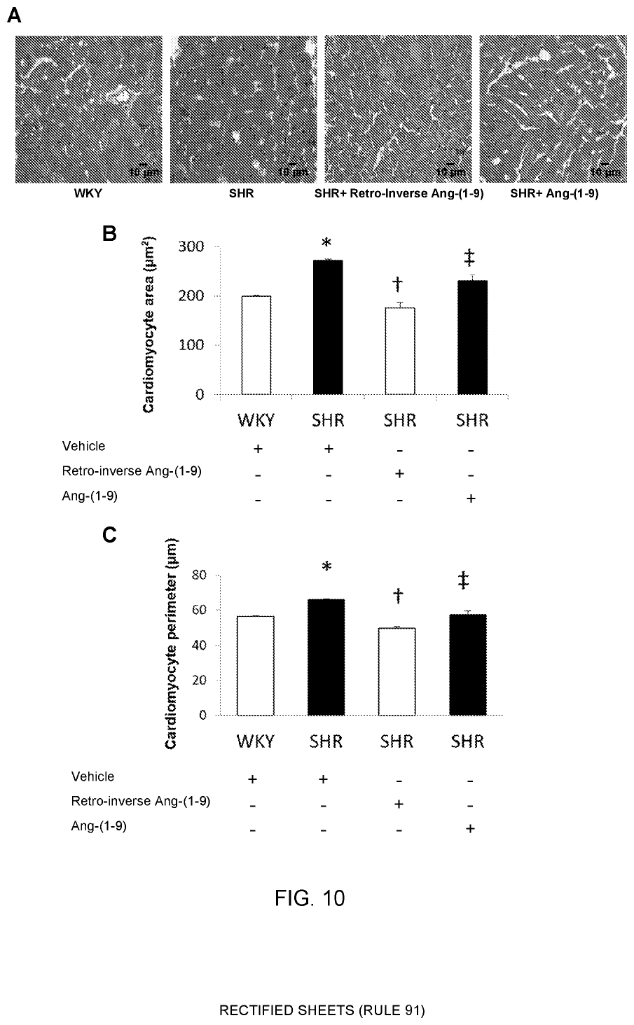

Measurement of cardiac hypertrophy by morphometry analysis. Morphometric analysis of the left ventricle was performed to assess cardiac hypertrophy. The hearts were washed in saline, weighed, and fixed in 4% formalin in PBS for 12 h and then embedded in paraffin. Fixed hearts were cut into transverse sections (5 μm) and stained with hematoxylin and eosin. Tissues were visualized by light microscopy. Cardiomyocyte size (area and perimeter) was determined as described by Ocaranza et al. (J. Hypertens. 2010; 28:1054-1064). As observed with the above cardiac hypertrophic marker, β-MHC, in hypertensive SHR, an increase in the area and perimeter of cardiomyocytes were found, confirming the occurrence of cardiac hypertrophy. The treatment with retro-inverse angiotensin-(1-9) reversed the increase of area and perimeter of cardiomyocytes induced by hypertension. These decreases are like those observed with angiotensin-(1-9) (FIGS. 10A, 10B, and 10C). Taken together, β-MHC and cardiomyocyte area and perimeter, indicates that retro-inverse angiotensin-(1-9) reverse cardiac hypertrophy induced by hypertension, and its effect was similar to those observed with angiotensin-(1-9).

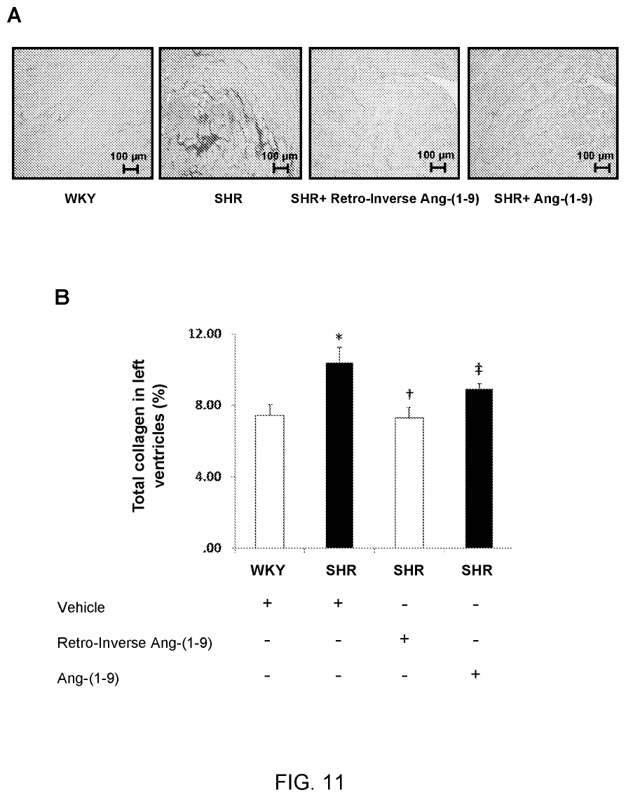

Evaluation of cardiac fibrosis. Morphometry of the left ventricle was performed to assess cardiac fibrosis. The hearts were washed in saline, weighed, and fixed in 4% formalin in PBS for 12 h and then embedded in paraffin. Fixed hearts were cut into transverse sections (5 μm) and stained with picrosirius red for the fibrosis assessment as described by Ocaranza et al (J. Hypertens. 2014; 32:771-783). In hypertense SHR increased picrosirius red staining was detected in the hearts, indicating the presence of interstitial collagen, a marker of cardiac fibrosis. The treatment with retro-inverse angiotensin-(1-9) completely revert the accumulation of collagen in cardiac tissue. This effect is similar to that obtained by using angiotensin-(1-9) (FIGS. 11A and 11B). These results indicates that retro-inverse angiotensin-(1-9) reverts cardiac fibrosis induced by hypertension.

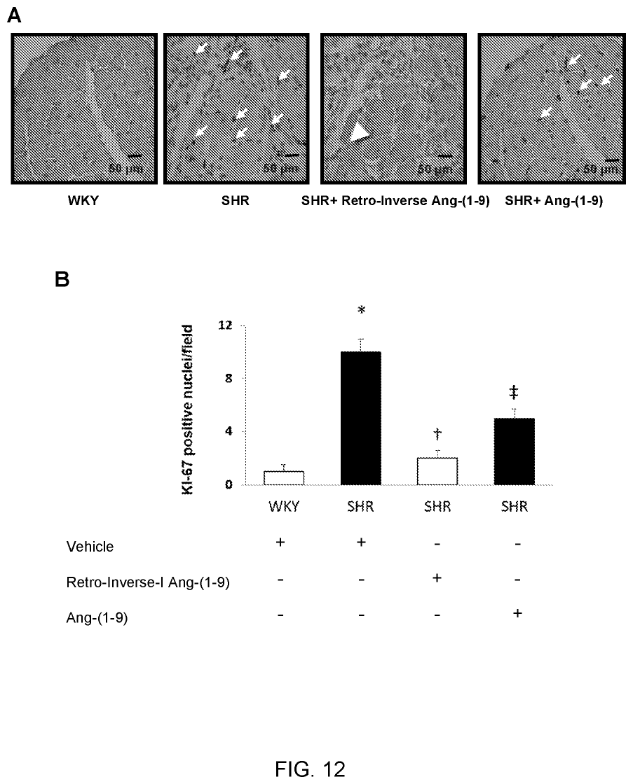

Immunohistochemistry for Ki67. Proliferation of fibroblast, another marker of fibrosis, was assessed by measuring the presence of Ki-67 positive nuclei. Transverse sections (5 μm) of heart slices were fixed and embedded in paraffin. The sections were later deparaffinized, hydrated, and denatured to expose the antigen with 1 mM EDTA at pH 8.0. Immunostaining was performed with a K0679 DAKO kit (Agilent Technologies Inc., Santa Clara, CA, USA). Sections were immunostained for Ki67 with monoclonal IgG antibodies (1/50, Dako) and with a biotinylated anti-mouse IgG (Dako). Sections were then developed by using 3,3 diaminobenzidine (Dako) as the chromogen, and counterstained with hematoxylin. Cells with brown granules in the nucleus were positive cells. Two blinder researchers counted manually positive cells in 10 high-power fields (×40). When the results were inconsistent between the two researchers, the slide was reexamined. The percentage of positive Ki67 cells was calculated. As observed with the above cardiac fibrosis marker, collagen content assessed by picrosirius red staining, in hypertensive SHR, an increase in fibroblast with Ki-67 positive nuclei were found, confirming the occurrence of cardiac fibrosis. The treatment with retro-inverse angiotensin-(1-9) reversed the increase of fibroblast with Ki-67 positive nuclei induced by hypertension. These decreases are like those observed with angiotensin-(1-9) (FIGS. 12A and 12B). Taken together, collagen content, assessed by picrosirius red staining, and fibroblast proliferation, assessed by the presence of Ki-67 positive nuclei, indicates that retro-inverse angiotensin-(1-9) reverse cardiac fibrosis induced by hypertension, and its effect was similar to those observed with angiotensin-(1-9).

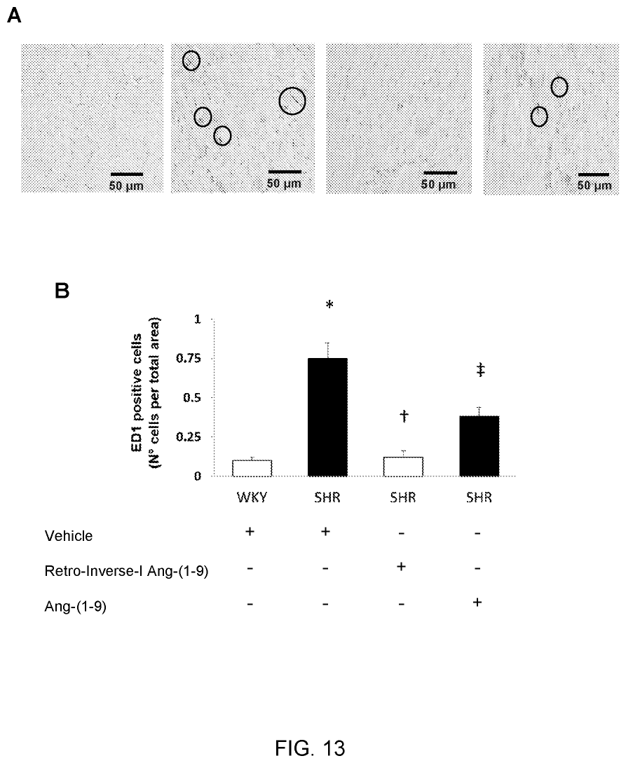

Evaluation of cardiac inflammation by monocyte infiltration. Monocyte infiltration was assessed as a marker of cardiac inflammation. Monocytes were detected by immunohistochemistry using ED-1. Transverse sections (4 μm) of left ventricles were fixed and embedded in paraffin. The sections were later deparaffinized, hydrated, and denatured to expose the antigen with 1 mM EDTA at pH 8.0. Immunostaining was performed with a K0679 DAKO kit. The sections were incubated with an antibody against the rat monocyte/macrophage antigen (ectodermal dysplasia (ED1, Serotec MCA341R) in a 1:200 dilution overnight at 4° C. in a humid chamber. Subsequently, the cardiac tissues were washed and incubated with a biotinylated secondary antibody for 30 min at room temperature. The diaminobenzidine technique was used (DAKO kit) for detection. Samples were counter-stained with hematoxylin. The proportion of ED-1-positive cells was determined by evaluating the ratio between the total number of ED1-positive cells and the total area of the cardiac tissue (ED-1(+) cells/mm²). In hypertense SHR increased ED-1 positive cells was detected in the hearts, indicating the presence of monocyte infiltration, a marker of cardiac inflammation. The treatment with retro-inverse angiotensin-(1-9) completely revert the accumulation of ED-1 positive cells in cardiac tissue. This effect is similar to that obtained by using angiotensin-(1-9) (FIGS. 13A and 13B). These results indicates that retro-inverse angiotensin-(1-9) reverts cardiac inflammation induced by hypertension.

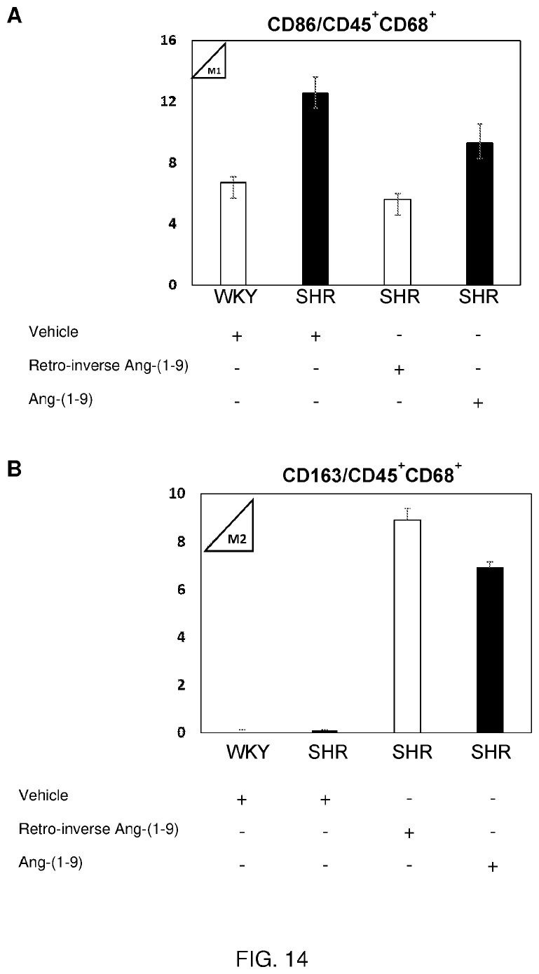

Evaluation of cardiac inflammation by cardiac-resident macrophage phenotype. Macrophages can display two phenotypes: M1 proinflammatory and M2 anti-inflammatory. Evaluation of cardiac resident macrophages phenotypes can be used to assess cardiac tissue inflammatory status. Cardiac tissues were digested with a solution containing 125 U/mL Collagenase Type XI, 60 U/mL Hyaluronidase type I, 60 U/mL DNase I, 450 U/mL Collagenase Type I, 20 mM HEPES in 1× Phosphate Buffered Saline (PBS) as previously described by Allen et al J Vis Exp. 2017; (122).55445). M1 and M2 macrophages were identified by flow cytometry using CD45+CD68+CD86+ and CD45+CD68+CD163+ as described by Moore et al. (J Immunol Methods. 2013; 396:33-43). As observed with the above cardiac inflammation marker, monocyte infiltration in cardiac tissue, in hypertensive SHR, an increase in macrophages M1 and an absence of macrophages M2 were found, confirming the occurrence of cardiac inflammation. The treatment of SHR with retro-inverse angiotensin-(1-9) reversed the increase of macrophages M1 and increases the macrophages M2 in cardiac tissues. In This case, retro-inverse angiotensin-(1-9) produced a more marked decrease in macrophages M1 than angiotensin-(1-9), but with a similar increase of macrophages M2 (FIGS. 14A and 14B). Taken together, monocyte infiltration, assessed by ED1 staining, and macrophage M1/M2 phenotype, indicates that retro-inverse angiotensin-(1-9) reverse cardiac tissue inflammation induced by hypertension, and its effect was more similar to those observed with angiotensin-(1-9).

Statistical analysis. Each experimental group contained 12 animals. Data are expressed as mean±S.E.M. Comparisons were performed using ANOVA and Newman-Keuls post-tests. A p<0.05 was considered statistically significant.

1. A peptide comprising or consisting of the amino acid sequence HFPHIYVRD (SEQ ID NO: 9), wherein the peptide comprises one or more D-amino acid residues.

2. The peptide according to claim 1, wherein the peptide comprises or consists of the sequence NH₂-^{D}H^{D}F^{D}P^{D}H^{D}I^{D}Y^{D}V^{D}R^{D}D-COOH (SEQ ID NO: 10).

3. The peptide according to claim 1, wherein the peptide comprises or consists of the sequence NH₂-^{D}H^{D}F^{D}P^{D}H^{D}I^{D}Y^{D}V^{D}R^{D}D-CONH₂ (SEQ ID NO: 11).

4. A pharmaceutical composition comprising an effective amount of the peptide of claim 1, and at least one pharmaceutically acceptable carrier, excipient, stabilizer, diluent, and/or adjuvant.

5. The pharmaceutical composition according to claim 4, in a liquid or solid form, for combined administration to a subject concomitantly with at least one further pharmaceutical compound.

6. (canceled)

7. The pharmaceutical composition according to claim 5, wherein the further pharmaceutical compound is selected from angiotensin 1-converting enzyme inhibitor, angiotensin II receptor antagonist (AT1), L-calcium channel antagonist, Rho kinase inhibitor, diuretic, renin inhibitors or other retro-inverse peptides.

8. The pharmaceutical composition according to claim 7, wherein such angiotensin 1-converting enzyme inhibitor is any drug selected from lisinopril, enalapril, captopril, zofenopril, ramipril, quinapril, perindopril, benazepril and fosinopril; angiotensin II receptor antagonist (AT1) is any drug selected from valsartan, telmisartan, losartan, irbesartan, olmesartan, candesartan, eprosartan and saralasine; L-calcium channel antagonist is any drug selected from nicardipine, nifedipine, amlodipine, felodipine, nitrendipine, nisoldipine, isradipine, nimodipine, diltiazem, clentiazem, verapamil, gallopamil, anipamil, RO5967, falipamil; Rho kinase inhibitor is any drug selected from fasudil, hydroxyl-fasudil, 3-(4-piridil)-1H-indol, (S)-(+)-2-methyl-1-[(4-methyl-5-isoquinolinyl)sulfonyl]homopiperazine, N-(4-piridyl)-N′-(2,4,6-trichlorophenyl) urea; diuretic is any drug selected from bendroflumethiazide, benzithiazide, chlorothiazide, chlortalidone, hydrochlorothiazide, hydroflumethiazide, indapamide, metyclothiazide, metolazone, polythiazide, kinetazone, trichlormethiazide, xipamide, furosemide, torasemide, bumetanide, ethacrynic acid, acetazolamide, dorzolamide, amiloride, triamterene, spironolactone, canrenoate, eplerenone, and mannitol; renin inhibitor is any drug selected from pepstatin, CGP2928, remikiren, enalkiren, zankiren, aliskiren; and other retro-inverse peptide is any drug selected from retro-inverse bradykinin and retro-inverse AT1R.

9. The pharmaceutical composition according to claim 4, wherein the pharmaceutical composition is suitable for administration via injection, parenteral administration, inhalation, continuous release, a continuous release pump, a suppository, or oral administration.

10. Use of the peptide according to claim 1, or the pharmaceutical composition according to claim 4, for the manufacture of a medicament for treating a cardiovascular disease, wherein the cardiovascular disease is selected from heart failure (congestive heart failure, compensated heart failure, decompensated heart failure, and the like), restenosis, hypertension (low-renin hypertension; salt-sensitive hypertension; low-renin, salt-sensitive hypertension; primary pulmonary hypertension; thromboembolic pulmonary hypertension; pregnancy-induced hypertension; renovascular hypertension), heart hypertrophy, diastolic dysfunction, coronary artery disease, myocardial infarctions, cerebral infarctions, atherosclerosis, atherogenesis, cerebrovascular disease, angina, (including chronic, stable, unstable and variant (Prinzmetal) angina pectoris), aneurysm, ischemic heart disease, cerebral ischemia, myocardial ischemia, thrombosis, platelet aggregation, platelet adhesion, smooth muscle cell proliferation, vascular or non-vascular complications associated with the use of medical devices, wounds associated with the use of medical devices, vascular or non-vascular wall damage, peripheral vascular disease, neointimal hyperplasia following percutaneous transluminal coronary angiograph, vascular grafting, coronary artery bypass surgery, thromboembolic events, post-angioplasty restenosis, coronary plaque inflammation, hypercholesterolemia, embolism, stroke, shock, arrhythmia, atrial fibrillation or atrial flutter, thrombotic occlusion and reclusion cerebrovascular incidents, left ventricular dysfunction and hypertrophy, and the like.

11. (canceled)

12. Use of the peptide according to claim 1, or the pharmaceutical composition according to claim 4, for the manufacture of a medicament for preventing, reverting, inhibiting, and/or reducing cardiovascular, pulmonary, renal, and/or cerebral remodeling, wherein cardiovascular remodeling comprises cardiac fibrosis, cardiomyocyte hypertrophy, cardiovascular inflammation, and/or fibroblast proliferation.

13. (canceled)

14. Use of the peptide according to claim 1, or the pharmaceutical composition of according to claim 4, for the manufacture of a medicament for inducing cardioprotection, and/or decreasing myocardial infarct size.

15. (canceled)

16. Method for treating a cardiovascular disease, comprising administering a therapeutically effective amount of the peptide according to claim 1, or the pharmaceutical composition according to claim 4, to a subject in need thereof, wherein the method further comprises raising a concentration of the peptide in the blood and/or tissues, particularly plasma, heart, lung, kidney, brain and/or vascular bed of the subject.

17. Method for preventing, reversing, inhibiting and/or decreasing cardiovascular, renal, pulmonary, and cerebral remodeling, comprising administering a therapeutically effective amount of the peptide according to claim 1, or the pharmaceutical composition according to claim 4, to a subject in need thereof, wherein the method further comprises raising a concentration of the peptide in the blood and/or tissues, particularly plasma, heart, lung, kidney, brain and/or vascular bed of the subject.

18. Method for inducing cardioprotection, comprising administering a therapeutically effective amount of the peptide according to claim 1, or the pharmaceutical composition according to claim 4, to a subject in need thereof, wherein the method further comprises raising a concentration of the peptide in the blood and/or tissues, particularly plasma, heart, lung, kidney, brain and/or vascular bed of the subject.

19. (canceled)


SEQUENCE LISTING (11 sequences)

SEQ ID NO: 1 - protein, 14 aa, Homo sapiens
DRVYIHPFHLLVYS

SEQ ID NO: 2 - protein, 10 aa, Homo sapiens
DRVYIHPFHL

SEQ ID NO: 3 - protein, 8 aa, Homo sapiens
DRVYIHPF

SEQ ID NO: 4 - protein, 7 aa, Homo sapiens
RVYIHPF

SEQ ID NO: 5 - protein, 6 aa, Homo sapiens
VYIHPF

SEQ ID NO: 6 - protein, 9 aa, Homo sapiens
DRVYIHPFH

SEQ ID NO: 7 - protein, 7 aa, Homo sapiens
DRVYIHP

SEQ ID NO: 8 - protein, 9 aa, Artificial Sequence
  Retro angiotensin-(1-9) (D-amino acids, N to C direction)
  SITE (1)..(1): D-aspartic acid
  SITE (2)..(2): D-arginine
  SITE (3)..(3): D-valine
  SITE (4)..(4): D-tyrosine
  SITE (5)..(5): D-isoleucine
  SITE (6)..(6): D-histidine
  SITE (7)..(7): D-proline
  SITE (8)..(8): D-phenylalanine
  SITE (9)..(9): D-histidine
DRVYIHPFH

SEQ ID NO: 9 - protein, 9 aa, Artificial Sequence
  Inverse angiotensin-(1-9) (L-amino acids, N to C direction)
HFPHIYVRD

SEQ ID NO: 10 - protein, 9 aa, Artificial Sequence
  Retro-inverse of angiotensin-(1-9) (D-amino acids, N to C direction)
  SITE (1)..(1): D-histidine
  SITE (2)..(2): D-phenylalanine
  SITE (3)..(3): D-proline
  SITE (4)..(4): D-histidine
  SITE (5)..(5): D-isoleucine
  SITE (6)..(6): D-tyrosine
  SITE (7)..(7): D-valine
  SITE (8)..(8): D-arginine
  SITE (9)..(9): D-aspartic acid
HFPHIYVRD

SEQ ID NO: 11 - protein, 9 aa, Artificial Sequence
  Retro-inverse angiotensin-(1-9) amide
  SITE (1)..(1): D-histidine
  SITE (2)..(2): D-phenylalanine
  SITE (3)..(3): D-proline
  SITE (4)..(4): D-histidine
  SITE (5)..(5): D-isoleucine
  SITE (6)..(6): D-tyrosine
  SITE (7)..(7): D-valine
  SITE (8)..(8): D-arginine
  SITE (9)..(9): D-aspartic acid
  MOD_RES (9)..(9): AMIDATION (C-terminal amide group)
HFPHIYVRD